SEQUENCE LISTING (14 sequences)

SEQ ID NO: 1 - DNA, 20 bp, synthetic construct
  misc_feature 1..20: note = Synthetic primer
  source 1..20: mol_type = other DNA
atacccatggccaacctcct

SEQ ID NO: 2 - DNA, 20 bp, synthetic construct
  misc_feature 1..20: note = Synthetic primer
  source 1..20: mol_type = other DNA
gggcctttgcgtagttgtat

SEQ ID NO: 3 - DNA, 19 bp, synthetic construct
  misc_feature 1..19: note = Synthetic primer
  source 1..19: mol_type = other DNA
tgacccaccaatcacatgc

SEQ ID NO: 4 - DNA, 20 bp, synthetic construct
  misc_feature 1..20: note = Synthetic primer
  source 1..20: mol_type = other DNA
atcacatggctaggccggag

SEQ ID NO: 5 - DNA, 20 bp, synthetic construct
  misc_feature 1..20: note = Synthetic primer
  source 1..20: mol_type = other DNA
cccgcagccgagccgcgggg

SEQ ID NO: 6 - DNA, 27 bp, synthetic construct
  misc_feature 1..27: note = Synthetic primer
  source 1..27: mol_type = other DNA
tcttccaactgcctctctggccctccg

SEQ ID NO: 7 - DNA, 20 bp, synthetic construct
  misc_feature 1..20: note = Synthetic primer
  source 1..20: mol_type = other DNA
tgcaccaccaactgcttagc

SEQ ID NO: 8 - DNA, 21 bp, synthetic construct
  misc_feature 1..21: note = Synthetic primer
  source 1..21: mol_type = other DNA
ggcatggactgtggtcatgag

SEQ ID NO: 9 - DNA, 21 bp, synthetic construct
  misc_feature 1..21: note = Synthetic primer
  source 1..21: mol_type = other DNA
catgtacgttgctatccaggc

SEQ ID NO: 10 - DNA, 21 bp, synthetic construct
  misc_feature 1..21: note = Synthetic primer
  source 1..21: mol_type = other DNA
ctccttaatgtcacgcacgat

SEQ ID NO: 11 - DNA, 24 bp, synthetic construct
  misc_feature 1..24: note = Synthetic: mtDNA Forward
  source 1..24: mol_type = other DNA
cccagctactaccatcattcaagt

SEQ ID NO: 12 - DNA, 25 bp, synthetic construct
  misc_feature 1..25: note = Synthetic: mtDNA Reverse
  source 1..25: mol_type = other DNA
gatggtttgggagattggttgatgt

SEQ ID NO: 13 - DNA, 22 bp, synthetic construct
  misc_feature 1..22: note = Synthetic: nuDNA Forward
  source 1..22: mol_type = other DNA
tttgctcctgggcctccaagtt

SEQ ID NO: 14 - DNA, 22 bp, synthetic construct
  misc_feature 1..22: note = Synthetic: nuDNA Reverse
  source 1..22: mol_type = other DNA
agcccgtgactgccacaaatca

(19) United States
(12) Patent Application Publication
(10) Pub. No.: US 2024/0102995 A1
(43) Pub. Date: Mar. 28, 2024

(54) Fluorescence Detection of Circulating Cell Free DNA Including Within Extracellular Vesicles in Biospecimens and Liquid Biopsies

(71) Applicants: BOARD OF REGENTS, THE UNIVERSITY OF TEXAS SYSTEM, Austin, TX (US); KANSAS STATE UNIVERSITY RESEARCH FOUNDATION, Manhattan, KS (US)

(72) Inventors: Bartosz Szczesny, Galveston, TX (US); Massoud Motamedi, Galveston, TX (US); Stefan H. Bossmann, Manhattan, KS (US)

(21) Appl. No.: 18/468,020

(22) Filed: Sep. 15, 2023

Related U.S. Application Data
(63) Continuation-in-part of application No. PCT/US2022/020316, filed on Mar. 15, 2022.
(60) Provisional application No. 63/161,151, filed on Mar. 15, 2021.

(51) Int. Cl. G01N 33/53

(52) U.S. Cl. CPC G01N 33/5308

(57) ABSTRACT

The present invention relates in general to the field of rapid detection and quantification of tissue/organ injury, and more particularly, to a novel platform for monitoring and quantification of circulating cell-free DNA including within extracellular vesicles, together with extracellular vesicles specific markers, and extracellular vesicles sizes also in combination with PCR-related technologies in biospecimens, and liquid biopsies for the assessment and prediction of severity of tissue/organ injury, monitoring of disease progression as well as the assessment of the response to therapeutic interventions.

### CROSS-REFERENCE TO RELATED APPLICATIONS

This application claims priority to and is a continuation-in-part of International Application No. PCT/US2022/020316, filed Mar. 15, 2022, which claims priority to U.S. Provisional Application Ser. No. 63/161,151, filed Mar. 15, 2021, the entire contents of which are incorporated herein by reference.

### STATEMENT OF FEDERALLY FUNDED RESEARCH

This invention was made with government support under 1804422, 1804416 and 2136421 awarded by the National Science Foundation. The government has certain rights in the invention.

### TECHNICAL FIELD OF THE INVENTION

The present invention relates in general to the field of rapid detection and quantification of tissue/organ injury, and more particularly, to a novel platform for monitoring and quantification of circulating cell free DNA (ccf-DNA) including within extracellular vesicles (EVs) and mitochondrial derived vesicles (collectively called EVs), including EVs sizes and their specific markers, in biospecimens, and liquid biopsies for the assessment and prediction of severity of tissue/organ injury, monitoring of disease progression as well as the assessment of the response to therapeutic interventions.

### INCORPORATION-BY-REFERENCE OF MATERIALS FILED ON COMPACT DISC

The present application includes a Sequence Listing which has been submitted in XML format via EFS-Web and is hereby incorporated by reference in its entirety. Said XML copy, created on Nov. 22, 2023, is named UTMB1064CIP.xml and is 18,937 bytes in size.

### BACKGROUND OF THE INVENTION

Without limiting the scope of the invention, its background is described in connection with detection and quantification of tissue/organ injury by monitoring changes of the circulating cell free DNA alone or in combination with other key markers of tissue injury including markers of EVs or in combination with other PCR-related technologies that can be detected and quantified in liquid biopsy samples.

The analysis of circulating cell free DNA (ccf-DNA) is an emerging diagnostic tool for the detection and monitoring of disease progression, and potential therapy effects. Currently, virtually all ccf-DNA in tissue and liquid biopsies is analyzed with real-time quantitative PCR (qPCR) that is primer-specific, template-dependent, labor intensive and cost-inefficient and not suitable for integration with a point of care device engineered for rapid, cost effective office based or field-based diagnosis and monitoring.

Thus, a need exists for a more rapid, scalable, and less expensive way to detect ccf-DNA in readily available in liquid biopsy.

### SUMMARY OF THE INVENTION

The present invention relates to novel platform for rapid detection and quantification of tissue/organ injury, by monitoring and quantification of circulating cell-free DNA (ccf-DNA) including within extracellular vesicles including extracellular vesicles (EVs) derived from mitochondria (collectively called EVs) as well as by monitoring and quantification of EVs sizes, surface features, contents and their specific markers, in biospecimens, and liquid biopsies for the assessment and prediction of severity of tissue/organ injury, monitoring of disease progression as well as the assessment of the response to therapeutic interventions. These could be obtained using described herein novel platform for rapid detection of ccf-DNA with DNA-specific fluorescent probes and extended for identification of cell/tissue/organ specific markers present in EVs containing ccf-DNA and in combination with other PCR-related technologies.

As embodied and broadly described herein, an aspect of the present disclosure relates to a method for detecting DNA including mitochondrial DNA (mtDNA) in a biological sample comprising: obtaining the biological sample; and detecting the presence of DNA including mtDNA in the sample by staining EVs in the same with a DNA staining dye. In one aspect, the DNA staining dye is PicoGreen. In another aspect, the biological sample is a liquid biopsy. In another aspect, the mtDNA is detected without mtDNA specific primers or template-dependence. In another aspect, the DNA is ccf-DNA, or DNA in EVs. In another aspect, a DNA sample is obtained and tested single or multiple times in an emergency room, a sideline, a locker room, or a battlefield. In another aspect, the biological sample is obtained from a patient suspected of having a lung adenocarcinoma. In another aspect, an amount of mtDNA in the sample is increased when compared to a sample obtained from a subject not having a disease. In another aspect, the biological sample is obtained from a patient suspected of having a brain trauma, a concussion, or acute illness with a change in oxidative stress. In another aspect, the biological sample is obtained from a patient having or suspected of having SARS-CoV-2 or other virus infection. In another aspect, the biological sample is obtained from a person exposed to toxins or chemicals known to induce acute injury such as respiratory failure or chemical burn. In another aspect, the biological sample is obtained from a patient suffering from drug overdose. In another aspect, the biological sample is from a subject suspected of having a traumatic brain injury and one or more proteins in the isolated EVs from plasma is/are up-regulated: serum amyloid A (SAA); complement factor D (CFD), Corticosteroid-binding globulin (Q06770); Multimerin-1 (B2RPV6); Kininogen-1; Kininogen-1 heavy chain; Bradykinin; Kininogen-1 light chain (O08677); Proteasome subunit beta type-5 (O55234); Coagulation factor X; Factor X light chain; Factor X heavy chain; Activated factor Xa heavy chain (O88947); Afamin (O89020; O89020-3; O89020-2); Carbonic anhydrase 2 (P00920); Ig gamma-2A chain C region secreted form (P01864); Hemoglobin subunit alpha (P01942); Hemoglobin subunit beta-1;Hemoglobin subunit beta-2 (P02088; P02089); Complement factor D (P03953-2; P03953); Complement factor B; Complement factor B Ba fragment; Complement factor B Bb fragment (P04186); Fructose-bisphosphate aldolase A (P05064); L-lactate dehydrogenase A chain (P06151); Complement component C9 (P06683); Apolipoprotein A-IV (P06728); Transthyretin (P07309); Serum albumin (P07724); Alpha-1-antitrypsin 1-1 (P07758); Serine protease inhibitor A3K (P07759); Apolipoprotein A-II; Proapolipoprotein A-II (P09813); Major urinary protein 1 (P11588); Gelsolin (P13020-2; P13020); Glyceraldehyde-3-phosphate dehydrogenase (P16858); Prothrombin; Activation peptide fragment 1; Activation peptide fragment 2; Thrombin light chain; Thrombin heavy chain (P19221); 78 kDa glucose-regulated protein; Heat shock-related 70 kDa protein 2; Heat shock 70 kDa protein 1-like (P20029; P17156; P16627); Vitamin D-binding protein (P21614); Alpha-1-antitrypsin 1-2 (P22599); Carboxylesterase 1C (P23953); Talin-1 (P26039); Murinoglobulin-1 (P28665); Alpha-2-HS-glycoprotein (P29699); Vitronectin (P29788); Antithrombin-III (P32261); Leukemia inhibitory factor receptor (P42703-2; P42703); Pyruvate kinase PKM (P52480-2; P52480); Actin, cytoplasmic 2; Actin, cytoplasmic 2, N-terminally processed; Actin, cytoplasmic 1; Actin, cytoplasmic 1, N-terminally processed; Actin, gamma-enteric smooth muscle; Actin, alpha skeletal muscle; Actin, alpha cardiac muscle 1; Actin, aortic smooth muscle; Beta-actin-like protein 2 (P63260; P60710; P63268; P68134; P68033; P62737; Q8BFZ3); Apolipoprotein A-I; Proapolipoprotein A-I; Truncated apolipoprotein A-I (Q00623); Retinol-binding protein 4 (Q00724); Alpha-1-antitrypsin 1-3 (Q00896); Alpha-1-antitrypsin 1-4 (Q00897); Alpha-1-antitrypsin 1-5 (Q00898); Epidermal growth factor receptor (Q01279); Beta-2-glycoprotein 1 (Q01339); Serine protease inhibitor A3M (Q03734); Apolipoprotein C-II (Q05020); Clusterin; Clusterin beta chain; Clusterin alpha chain (Q06890); Protein AMBP; Alpha-1-microglobulin; Inter-alpha-trypsin inhibitor light chain; Trypstatin (Q07456); Complement factor I; Complement factor I heavy chain; Complement factor I light chain (Q61129); Ceruloplasmin (Q61147); Peroxiredoxin-2 (Q61171); Alpha-2-antiplasmin (Q61247); Haptoglobin; Haptoglobin alpha chain; Haptoglobin beta chain (Q61646); Inter-alpha-trypsin inhibitor heavy chain H3 (Q61704); Zinc-alpha-2-glycoprotein (Q64726); Complement component C8 beta chain (Q8BH35; Q8BH35-2); Sulfhydryl oxidase 1 (Q8BND5-3; Q8BND5-2; Q8BND5); EGF-containing fibulin-like extracellular matrix protein 1 (Q8BPB5); Complement C1r-A subcomponent; Complement C1r-A subcomponent heavy chain; Complement C1r-A subcomponent light chain; Complement C1r-B subcomponent; Complement C1r-B subcomponent heavy chain; Complement C1r-B subcomponent light chain (Q8CG16; Q8CFG9); Complement component C8 alpha chain (Q8K182); Complement component C8 gamma chain (Q8VCG4); Hemopexin (Q91X72); Serotransferrin (Q921I1); Vitamin K-dependent protein Z (Q9CQW3); Carboxypeptidase N subunit 2 (Q9DBB9); Inhibitor of carbonic anhydrase (Q9DBD0); Fetuin-B (Q9QXC1); Hepatocyte growth factor activator; Hepatocyte growth factor activator short chain; Hepatocyte growth factor activator long chain (Q9R098); Glycogen phosphorylase, muscle form (Q9WUB3); Platelet factor 4 (Q9Z126); or Aspartyl aminopeptidase (Q9Z2W0). In another aspect, the biological sample is from a subject suspected of having a traumatic brain injury and one or more proteins in the isolated EVs from plasma is/are down-regulated: haptoglobin (Hp); von Willebrand factor (VWF); Corticosteroid-binding globulin (Q06770); Coagulation factor XIII B chain (Q07968); Alpha-1B-glycoprotein (Q19LI2); Adiponectin (Q60994); Alpha-2-macroglobulin-P (Q6GQT1); Coagulation factor XIII A chain (Q8BH61); Fibrinogen beta chain; Fibrinopeptide B; Fibrinogen beta chain (Q8K0E8); Fibrinogen gamma chain (Q8VCM7); CD5 antigen-like (Q9QWK4); Proteasome subunit alpha type-1 (Q9R1P4); or Proteasome subunit alpha type-5 (Q9Z2U1). In another aspect, the method further comprises measuring the expression levels of one or more proteins selected from: Orm1, Alpha-1-acid glycoprotein 1; APOA1, Apolipoprotein A1; Saa, Serum Amyloid A; Aldob, Aldolase; Serpina, Alpha-1-Antitrypsin; Cir1, Corepressor Interacting With RBPJ; Apoc3, Apolipoprotein C-III; Thbs, Thrombospondin; Psma4, Proteasome 20S Subunit Alpha 4; Hspa, HSP70; Rp17, Ribosomal Protein L7; C1, Complement C; Amy, Amylase; Mup, Major Urinary Protein; Lcat, Lecithin-Cholesterol Acyltransferase; Acta, Actin Alpha 2; F7, Coagulation Factor VII; Cat, Catalase; Ces3a, Carboxylesterase 3; Aldi, Aldehyde dehydrogenase; Prdx, Peroxiredoxinin, in the biological sample. In another aspect, the method further comprises applying an algorithm to the measured protein expression, the algorithm generating a traumatic brain injury score based on a comparison of the measured expression levels to reference levels, wherein the algorithm is selected from a machine learning algorithm, a clustering algorithm, a support vector machine, or combinations thereof; and identifying the subject with the higher traumatic brain injury score as having a higher clinical outcome score for traumatic brain injury. In another aspect, the method further comprises measuring an amount of SAA, Hp, VWF, CFD, and CBG and using an algorithm to determine a time since a traumatic brain injury.

As embodied and broadly described herein, an aspect of the present disclosure relates to a method of detecting DNA comprising: obtaining or having obtained a biological sample; and detecting the presence of DNA including mtDNA in the sample by staining EVs including mitochondria derived vesicles in the same with a DNA staining dye, without the use of mtDNA specific primers, or by monitoring EVs sizes, quantification of EVs sizes, or EVs sizes specific markers. In one aspect, the dye is selected from at least one of GeiRed, 10 EvaGreen, SYBR, PicoGreen and derivatives, TOTO, YOYO, 8080, POPO, JOJO, LOLO, SYTOX, POPRO, 80-PRO, YO-PRO, TO-PRO, JO-PRO, PO-PRO, LO-PRO, and combinations thereof. In another aspect, the dyes are engineered for recognition and quantification of mtDNA encapsulated within EVs. In another aspect, the biological sample is a liquid biopsy. In another aspect, the biological samples is exhaled breath condensate. In another aspect, the DNA is circulating cell-free DNA, or DNA encapsulated in EVs. In another aspect, a DNA sample is obtained and tested in an emergency room, a critical care setting, an ICU, a sideline, a locker room, or a battlefield. In another aspect, a DNA sample is obtained and tested in a point of care diagnosis or an at home diagnosis. In another aspect, the biological sample is obtained from a patient suspected of having a lung adenocarcinoma. In another aspect, the biological sample is obtained from a patient suspected of having a brain trauma, a concussion, or a disease of oxidative stress. In another aspect, the biological sample is obtained from a patient suspected of having SARS-CoV-2 or other virus infection. In another aspect, an amount of mitochondrial DNA in the sample is increased when compared to a sample obtained from a subject not having a disease. In another aspect, the detection of DNA is done in conjunction with other markers of tissue injury.

As embodied and broadly described herein, an aspect of the present disclosure relates to a kit for determining an amount of mtDNA is a biological sample comprising: a container to obtaining the biological sample; and a reagent for detecting the presence of mtDNA, in the sample by staining EVs in the same with a DNA staining dye, wherein the presence or an increase in mtDNA is indicative of a disease or condition. In one aspect, the dye is selected from at least one of GeiRed, 10 EvaGreen, SYBR, PicoGreen and derivatives, TOTO, YOYO, 8080, POPO, JOJO, LOLO, SYTOX, POPRO, 80-PRO, YO-PRO, TO-PRO, JO-PRO, PO-PRO, LO-PRO, and combinations thereof. In another aspect, the kit further comprises primers to detect the presence of cell free nuclear DNA (nuDNA) primers to detect the presence of mtDNA, or both. In another aspect, the DNA is detected within a microfluidic device. In another aspect, the microfluidic device separates components of the biological sample prior to staining the mtDNA with the dye.

As embodied and broadly described herein, an aspect of the present disclosure relates to a method for rapid detection and quantification of tissue/organ injury, comprising: monitoring and quantifying ccf-DNA within EVs, monitoring and quantification of EV sizes, and EV-specific markers, in a biospecimen or a liquid biopsy to assess severity of tissue/organ injury, monitoring of disease progression, or assessment of a response to one or more therapeutic interventions.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B show serum of lung adenocarcinoma subjects have high amount of ccf-DNA. Total DNA from serum of healthy volunteers (n=20), subjects with benign tumors (n=20) and with lung adenocarcinoma (n=57) were analyzed. The amount of mtDNA (FIG. 1A) and nuDNA (FIG. 1B) was quantified with qPCR using mt-COXIII and nu-GAPDH and nu-SIRT1 primers, respectively. The amount of DNA in 100 μl of serum was quantified and analyzed.

FIGS. 2A and 2B show a subject with all stages of lung adenocarcinoma have high amount of ccf-DNA. Total DNA from serum of healthy volunteers (n=20), human subjects with benign tumors (n=20) and with stage I (n=21), stage II (n=12), stage III (n=13) and stage IV (n=11) of lung adenocarcinoma were analyzed. The amount of mtDNA (FIG. 2A) and nuDNA (FIG. 2B) was quantified with qPCR using mt-COXIII and nu-GAPDH and nu-SIRT1 primers, respectively. The amount of DNA in 100 μl of serum was quantified and analyzed.

FIGS. 3A to 3D show that traumatic brain injury patients have high amount of ccf-DNA. Total DNA from plasma of healthy volunteers (n=20) and trauma (TBI) subjects (n=57) was analyzed. The amount of mtDNA (FIG. 3A) and nuDNA (FIG. 3C) was quantified with qPCR using mt-COXIII, mt-NADI and nu-ACTB and nu-SIRT1 primers, respectively. The comparison in amount of ccf-mtDNA using mt-COXIII (FIG. 3B) and mt-NADI (FIG. 3D) with ccf-nuDNA using nu-ACTB and nu-SIRT1 is shown. The amount of DNA in 100 μl of serum was quantified and analyzed.

FIGS. 4A and 4B show a high amount of ccf DNA in TBI and lung adenocarcinoma subjects measured using PicoGreen reagent. The amount of DNA in plasma of (FIG. 4A) in subjects with TBI (n=57) and (FIG. 4B) lung adenocarcinoma subjects (n=16) in comparison with healthy volunteers (n=20 and n=4, respectively) was measured using the PicoGreen reagent. * p<0.05, **** p<0.0001.

FIGS. 5A to 5C show that EVs contain majority of the ccf-DNA that has mitochondrial origin. (FIG. 5A) Serum contains active DNase/s. Changes in the fluorescent units of Λ DNA incubated with increasing volume of serum. (FIG. 5B) Ccf-DNA is DNase insensitive. Serum samples of three lung adenocarcinoma subjects were incubated with recombinant DNase and changes in fluorescent units are shown. (FIG. 5C) Ccf-DNA in localized within lumen of EVs. Comparison between input, serum without EVs (supernatant) and isolated EVs (pellet) of three subjects using mtDNA-specific primers (mt-COXIII). The amount of nuDNA was negligible.

FIGS. 6A to 6C show high amount of circulating cell free DNA in serum of COVID-19 samples. Comparison of ccf-DNA in control (n=20) and COVID-19 (n=25) samples were quantified with (A) PicoGreen, and with qPCR using (B) mtDNA and (C) nuDNA specific primers. ***p<0.0001, ** p<0.05 based on Mann-Whitney T test.

FIGS. 7A to 7C show PicoGreen staining of ccf-DNA as great prognostic of COVID-19 severity in comparison to qPCR. Comparison of ccf-DNA in control (n=20). And mild (n=6), moderate (n=14), critical (n=5) COVID-19 samples using (A) PicoGreen staining, (B) RT-qPCR with mtDNA-specific primers, and (C) qPCR using nuDNA-specific primers. *p<0.01, *** p<0.001, **** p<0.0001 based on 2way ANOVA multiple comparison.

FIGS. 8A to 8C show PicoGreen (8A) and two derivatives (FIG. 8B and FIG. 8C) for use with the present invention.

FIGS. 9A to 9H. Altered physical properties and enhanced DNA content of EVs during acute TBI phase. Effect of TBI on (FIG. 9A) number, (FIG. 9B) mean and (FIG. 9C) mode size measured by NTA. (FIG. 9D) Validation of circular shape and changes in the size of isolated with Transmission Electron Microscopy. Marker, 150 nm. (FIG. 9E) Quantification of the CD63, exosome marker, in EVs isolated using Western analysis. Example of analysis of EVs isolated from 2 animals in each experimental group. Quantification of (FIG. 9F) mtDNA and (FIG. 9G) nuDNA using qPCR. (FIG. 9H) Comparison of the amount of mtDNA in isolated EVs (pellet, p) and in plasma post EVs isolation (supernatant, s). Data based on 10 animals in each experiment group (N=10). * p<0.05, ** p<0.01, *** p<0.001, **** p<0.0001 based on one-way ANOVA with Dunnett correction for multiple comparisons to sham.

FIGS. 10A to 10G. Distinctive dynamics of neuronal, microglia, and astrocyte markers in EVs induced by TBI. (FIG. 10A) Examples of Western blot analysis of neuronal (NFL), astrocyte (GFAP) and microglia (Iba1) in isolated EVs from 2 animals per experimental group. Quantification of levels of (FIG. 10B) NFL, (FIG. 10C) Iba1 and (FIG. 10D) GFAP in isolated EVs post TBI. (FIG. 10E) Examples of Western blot analysis of microglia/macrophage (CD11b) and astrocyte (ACSA-2) derived microvesicles in isolated EVs from 2 animals in each experimental group. Quantification of levels of (FIG. 10F) CD11b and (FIG. 10G) ACSS2 in isolated EVs post TBI. Data based on 10 animals (N=10) in each experiment group. * p<0.05, ** p<0.01, *** p<0.001, based on one-way ANOVA with Dunnett correction for multiple comparisons to sham.

FIGS. 11A to 11C. Proteomic profiling of EVs uncovers promising novel TBI biomarkers. (FIG. 11A) Volcano plots showing proteomics data for each experimental group in comparison to sham controls. Dashed horizontal line shows the p-values cutoff (p<0.05). Two dashed vertical lines indicate up/down regulated for 2-fold cutoff. Red dots represent proteins that are downregulated and green dots that are upregulated meeting threshold of p<0.05 and <−2 or >2 fold change (FC). The most promising novel biomarkers are labelled as SAA, serum amyloid A; CFD complement factor D (CFD), CBG, corticosteroid-binding globulin; Hp haptoglobin; VWF von Willebrand factor. List of proteins (FIG. 11B) upregulated and (FIG. 11C) downregulated with p<0.05 and <−2 or >2 fold FC, respectively.

FIGS. 12A to 12C. Computational identification of TBI-specific biomarkers. (FIG. 12A) Analysis flow using STRING to build initial protein-protein interaction (PPI) network followed by Graph Neural Network (GNN)-based framework. (FIG. 12B) GNN enrichment analysis for potential biomarkers for 3 hpi. Each protein is highlighted as a node with interactions between proteins represented as connections. Sliding color scale represents the confidence level of interaction between proteins as calculated from GNN. (FIG. 12C) List of potential biomarkers ranked 1-6. Orm1, Alpha-1-acid glycoprotein 1; APOA1, Apolipoprotein A1; Saa, Serum Amyloid A; Aldob, Aldolase; Serpina, Alpha-1-Antitrypsin; Cir1, Corepressor Interacting With RBPJ; Apoc3, Apolipoprotein C-III; Thbs, Thrombospondin; Psma4, Proteasome 20S Subunit Alpha 4; Hspa, HSP70; Rp17, Ribosomal Protein L7; C1, Complement C; Amy, Amylase; Mup, Major Urinary Protein; Lcat, Lecithin-Cholesterol Acyltransferase; Acta, Actin Alpha 2; F7, Coagulation Factor VII; Cat, Catalase; Ces3a, Carboxylesterase 3; Aldi, Aldehyde dehydrogenase; Prdx, Peroxiredoxin.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

### Example 1

Detection of ccf-DNA in serum and plasma of healthy volunteers, and subjects with lung adenocarcinoma, traumatic brain injury, and COVID-19.

The present invention compared the amounts of ccf-DNA in serum and plasma of healthy volunteers, and subjects with lung adenocarcinoma, traumatic brain injury and COVID-19 using qPCR and quantitative PicoGreen fluorescence, both detecting double-stranded DNA. A significant increase of ccf-DNA in lung adenocarcinoma, traumatic brain injury and COVID-19 patients, compared to the group of healthy human subjects was found using both analytical methods. Surprisingly, the inventors found a direct correlation between PicoGreen fluorescence and qPCR, but only when mtDNA-specific primers were used. In sharp contrast, the analysis of blood samples with several nuclear DNA-specific primers resulted in the differences of several orders of magnitude. Moreover, the inventors found that the amount of ccf-DNA (mostly mtDNA) in serum of COVID-19 patients collected at the admission to the hospital and measured with PicoGreen reagent directly correlate with severity of COVID-19 assessed later by oxygen supplement requirement and serve as an early predictor of COVID-19 severity. A more detailed analysis of the location of ccf-DNA indicated that the majority of DNA is located within EVs and is mainly of mitochondrial origin The inventors concluded that, due to the presence of active DNases in the blood, the analysis of DNA within EVs yield more specific information that can be used to determine therapeutically relevant outcomes. Moreover, the present invention provides a fast and reliable fluorescent detection of ccf-DNA that is particularly useful in the settings of point of care use or emergency room settings when an immediate result may provide clinically useful information. In addition, cell/tissue/organ specific markers present within the internal content or on the surface of EVs, source of EVs and changes in EVs sizes yield desired diagnostic information that can be used for the assessment of the degree of injury/trauma in specific organ or based on relevant information derived from the source of EVs and thus the site of injury.

The present invention includes a validated and fast, primer and template independent and inexpensive method for the quantification of the total amount of ccf-DNA in blood using the PicoGreen reagent. Most importantly, the inventors found that quantification of the amount of ccf-DNA with PicoGreen and using qPCR with mtDNA-specific primers, but not using nuDNA-specific primers, is well correlated. The quantification of ccf-DNA using nuDNA-specific primers resulted in differences of orders of magnitude between different primers. It is noteworthy that the inventors found the majority of ccf-DNA encapsulated in EVs that provided protection from DNase/s and that DNA is of mitochondrial origin. This novel approach is particularly useful in emergency settings for the quick and inexpensive assessment of the total amount of ccf-DNA in various body fluids. The present invention demonstrated the ability of the proposed approach to yield results that are comparable to those obtained by running conventional qPCR measurements, which are significantly more time-consuming. The present invention of quick and inexpensive quantification and monitoring of the total amount of ccf-DNA present in various body fluids can also complemented with other known or currently under development biomarkers and/or clinically appropriate testing to increase its specificity including novel PCR-related technologies including those that are designed to be used as point of care instrument. Below are two examples of such approach.

Any number of platforms can be used with the present invention. For example, a single or multiple detection system can be used and quantification of markers of disease or injury. One example is a system using optical based detectors can be used to manage the detection and processing of one or more of the markers of injury disclosed in this application using optical fluid chambers suitable for integration with optical and electrical sensors. One such example is a microfluidic device that can be used for sorting of the samples while enabling optical sensing and visualization of the targeted optical signal originating from the whole sample or portion of the sample stored in arrays of fluidic chamber. The system can include optical sensors including cameras, an image acquisition device (e.g., CCD camera, CID camera, CMOS arrays, photographic devices) as well as a processor device for the acquisition and the processing of the data. The sensing and imaging acquisition device can be developed to capture and store spectral and morphological features of the samples, e.g., both spatially and temporally as needed for a single chamber or several microfluidic devices. A single, or a combination of optical sensing and imaging modalities, can be used to detect and quantify the targeted biomarkers that are present in the liquid biopsy samples including, e.g., conventional fluorescence and time resolved fluorescence measurements, absorption, and reflection measurements and optoacoustic measurements. Furthermore, external energy sources can also be used, such as, electrical, magnetic, or acoustic sources to drive and position the liquid sample in the desirable location enabling optical detection of the targeted markers.

Detection and the assessment of severity of traumatic brain injury (TBI) based on the analysis of total DNA using PicoGreen reagent can be complemented with the assessment of changes in S100 calcium binding protein B (S100), glial fibrillary acidic protein (GFAP), total and phosphorylated tau protein (T-tau, P-Tau), neuron specific enolase (NSE), neurofilament protein light (NFL) that are well established markers of brain injury, and/or with Glasgow Coma Scale (GCS) that stratifies patients into categories of mild, moderate and severe TBI and/or computed tomography (CT) that provide additional specificity and severity of cranial injury.

The assessment of ccf-DNA with PicoGreen reagent can be also complemented with currently available or biomarkers that are under in development for acute respiratory distress syndrome (ARDS) such as biomarkers for endothelial damage (e.g. angiopoetin 1/2, Ang-1/2), alveolar damage (e.g. surfactant protein D, SP-D), cytokines/chemokines (e.g. Il1β, Il-6, IL-8, TNFα) and fibrinolysis (e.g. plasminogen activator inhibitor-1, PAI-1) together with clinical assessments of ARDS including chest imaging and/or oxygenation level. Together, measurement of total DNA in blood or other liquid specimen with PicoGreen reagent together with other known or under development biomarkers/clinical testing including specific markers of EVs as well as EVs sizes can be used as additional valuable measurement for diagnosis, monitoring, risk stratification/prediction, treatment surveillance and adjustment of targeted therapy.

Increased Amount of ccf-DNA in Serum of Human Lung Adenocarcinoma Subjects. The inventors analyzed serum samples of a group of patients diagnosed with various stages of lung adenocarcinoma (n=57), and a group with benign tumors (n=20) that the inventors received from the Lung Cancer Biospecimen Resource Network (https://lungbio.sites.virginia.edu). The basic demographic information of both groups is provided in Table 1.

TABLE 1
Demography of patients with benign and
with lung adenocarcinoma tumors.

| | | benign | lung adenocarcinoma |
|---|---|---|---|
| | | n = 20 | n = 57 |
| gender | male | 11 | 26 |
| | female | 9 | 31 |
| age (years) | mean ± SD | 59 ± 10 | 67 ± 10 |
| | range | 42-73 | 33-92 |
| smoking | never | 6 | 7 |
| history | quit | 7 | 35 |
| | current | 7 | 15 |
| tumor stage | I | | 21 |
| | II | | 12 |
| | III | | 13 |
| | IV | | 11 |

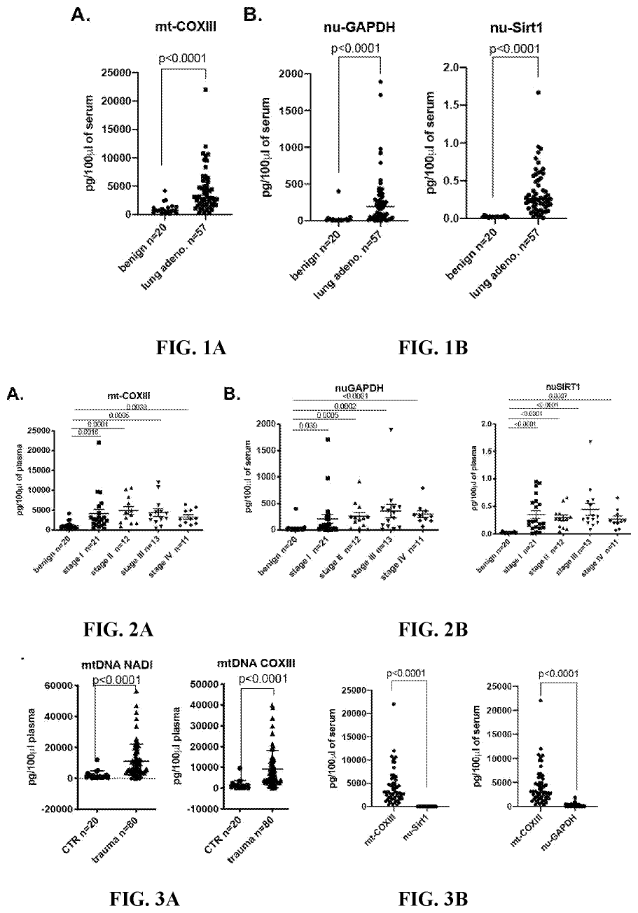

Total DNA in serum was isolated followed by RT-qPCR with three sets of primers: one located in the mitochondrial COXIII gene and two located in nuclear GAPDH and SIRT1 genes. The amount of DNA was calculated based on obtained ΔCt values, and expressed as pg of DNA in 100 μl of serum. These data were compared with the amount of DNA present in serum samples of healthy volunteers (n=20) using the same set of primers. The inventors did not find any differences in the amount of mtDNA between control, and subjects with benign tumors (FIG. 1A). However, the comparison of nuDNA in the control group and subjects with benign tumors shows an increase of nuDNA using nu-GAPDH primers, but a decrease with nu-SIRT1 primers (FIG. 1B). Next, the inventors compared the amount of ccf-DNA between the control group and subjects with benign tumors, with the human subjects that were diagnosed with lung adenocarcinoma. A significant increase of both mtDNA (mt-COXIII) and nuDNA (nu-GAPDH, nu-SIRT1) in subjects with lung adenocarcinoma was measured (FIGS. 1A, 1B). Interestingly, the inventors found a significant difference between the amount of the calculated DNA with mtDNA- and nuDNA-specific primers. Potentially, the high amount of mtDNA in comparison with nuDNA could be explained by the presence of the multiple mitochondria, with multiple copies of mtDNA in each cell. However, the difference of mtDNA and nuDNA (quantified with GAPDH- and SIRT1-specific primers) was found to be three orders of magnitude. This large difference suggests either unequal or specific representation of nuDNA in the serum, confirming primers and template dependence of RT-qPCR approach (Table 2).

TABLE 2
DNA in serum of subjects with lung adenocarcinoma (pg/100 μl)

| | mt-COXIII | nu-GAPDH | nu-Sirt1 |
|---|---|---|---|
| average | 4187.52 | 269.94 | 0.34 |
| SEM | 501.34 | 47.87 | 0.03 |

Next, the inventors analyzed mtDNA vs. nuDNA contents in the serum of the control group, of subjects with benign tumors, and of subjects with various stages of the lung adenocarcinoma. Regardless of the stage of lung adenocarcinoma (stages I-IV) the inventors measured increased amounts of both mtDNA (mt-COXIII) and nuDNA (nu-GAPDH, nu-SIRT1) when compared to members of the control group and subjects with benign tumors (FIG. 2A, 2B). No statistical differences in the amount of DNA were found when the inventors compared differences between various stages of lung adenocarcinoma using both mtDNA- or nuDNA-specific primers. The inventors found a significant increase of ccf-DNA in the serum of lung adenocarcinoma subjects is present. Surprisingly, the inventors also detected orders of magnitude differences in the amount of DNA when nuDNA-specific primers were used.

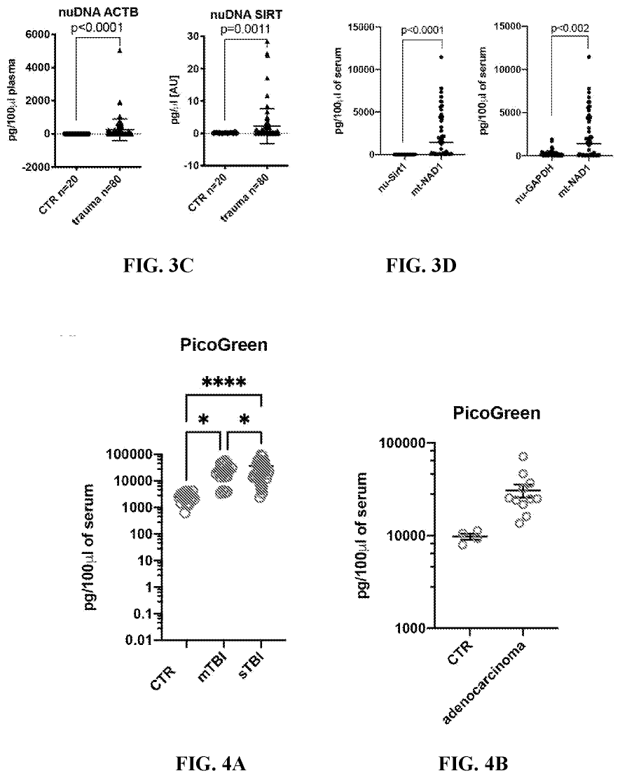

Increased amount of ccf-DNA in Plasma of Traumatic Brain Injury Subjects. The inventors analyzed plasma samples of healthy volunteers (n=20) and subjects with non-penetrating traumatic brain injury (TBI; n=57) that were admitted to the hospital of the University of Texas Health Science Center in Houston. Plasma of TBI subjects were harvested immediately post admission to the ICU unit. Similarly, as with the analysis of lung adenocarcinoma subjects, the total amount of DNA was analyzed using qPCR with mtDNA and nuDNA specific primers. The amount of mtDNA calculated with mt-COXIII- and mt-NADI specific primers was significantly increased in TBI subjects when compared to the control group (FIG. 3A). Since significant differences were observed in the amount of nuDNA quantified with two distinct set of primers in subjects with lung adenocarcinoma (FIG. 1B and Table 2), the inventors compared differences in the amount of mtDNA in controls and TBI subjects using mt-COXIII and mt-NADI primers. No significant changes were detected (FIG. 3B). The inventors measured a significant increase of nuDNA in TBI subjects when compared to the control group with both nuDNA-specific primers: nu-ACTB and nu-SIRT1 (FIG. 3C). These combined data clearly show an increased amount of ccf-DNA in serum of trauma subjects (here TBI) when compared to the healthy control group (FIG. 3D). Interestingly, the amount of mtDNA calculated using two different set of primers (mt-COXIII and mt-NADI) were nearly identical while the amount of nuDNA calculated with nu-ACTB and nu-SIRT1 was at least order of magnitude different.

Detection of ccf-DNA with PicoGreen Reagent. The data presented above clearly showed a significant increase of ccf-DNA that included both mtDNA and nuDNA, in human subjects diagnosed with lung adenocarcinoma or with TBI when compared to healthy controls. Our data also indicates that analysis of blood with qPCR can be primer and template specific, particularly for nuDNA. Based on this quantitative data obtained by qPCR, the inventors have developed a primer-unspecific, fast, and cost-effective method to detect ccf-DNA in blood samples. The reagent PicoGreen (λEx=480 nm/λEm=520 nm) can detect as little as 25 pg/ml of dsDNA in the presence of RNA, and free nucleotides. Whereas PicoGreen does not exhibit a significant background fluorescence signal, its fluorescence is switched on upon dsDNA binding (15). A recent study has demonstrated that PicoGreen acts as both, minor-groove binder and DNA intercalator (16). The assay is linear over three orders of magnitude (1 to 1000 ng dsDNA ml-1) and has no sequence dependence, thus allowing precision measurement of DNA in various liquid biopsies and tissue extracts. By using this assay, the inventors quantified the amount of ccf-DNA in 20 μl of plasma of healthy volunteers and human subjects with TBI (the same group as analyzed in FIG. 3), based on a standard curve that the inventors generated with Λ DNA. It should be noted that the PicoGreen assay works instantaneously, and that the fluorescence intensity remains constant after mixing. As quantified before with qPCR, significant increases of the ccf-DNA were detected in the TBI group when compared to their healthy counterparts (FIG. 4A). Interestingly, the inventors measured a 3.5-fold increase of the ccf-DNA in TBI subjects in average, using both the PicoGreen reagent (FIG. 4A) and RT-qPCR with mtDNA-specific primers (FIG. 3A). This is significantly different from the results obtained with RT-qPCR with nuDNA-specific primers (nu-ACTB and nu-SIRT1) where 200-fold and 15-fold increase of DNA was detected (FIG. 3C). Using the PicoGreen reagent the inventors established a low nanogram range for ccf-DNA (FIG. 4A), similar than with qPCR using mtDNA-specific primers (FIG. 3A) where low picogram ccf-DNA were calculated using qPCR with nuDNA-specific primers (FIG. 3C). These results clearly indicate that the major fraction of the ccf-DNA is of mitochondrial origin. It should be noted that the data obtained up to this point of our study were generated using serum/plasma samples that were stored for an extended period of time (years), which may affect the stability of DNA. In order to establish or eliminate a potential storage effect at −80° C. on ccf-DNA stability, the inventors obtained fresh blood of healthy volunteers (n=5) and subjects diagnosed with lung adenocarcinoma (n=16) from UTMB's clinic. In agreement with the results obtained with qPCR (FIGS. 1, 2), a significant increase amount of ccf-DNA was detected in the plasma of lung adenocarcinoma subjects when compared to the control group using the PicoGreen reagent (FIG. 4B). Based on this data, the inventors have established that the measurement of ccf-DNA using the PicoGreen reagent closely resembles data obtained with qPCR when mtDNA-specific primers were used. Furthermore, the inventors have shown that principally the same results can be obtained with fresh blood and blood serum or plasma that was stored at −80° C.

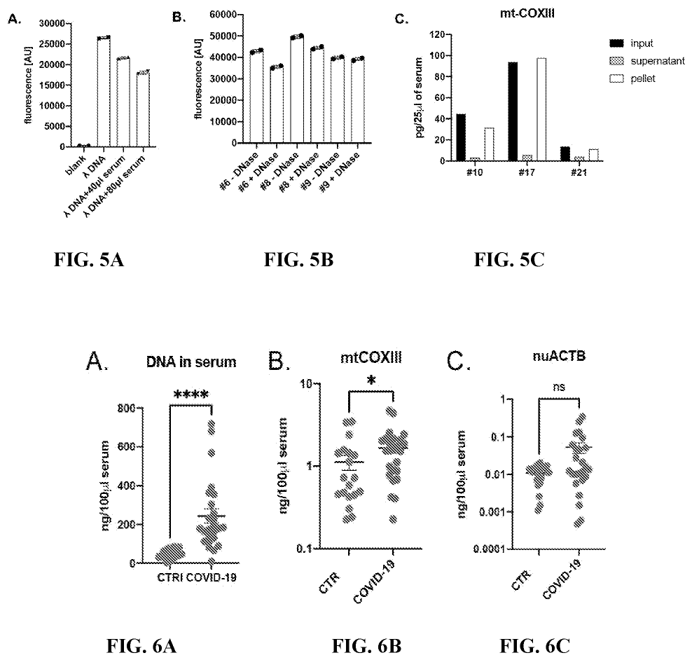

Ccf-DNA in Blood is Present within EVs. One of the major concerns that has to be taken under consideration during measurements of DNA concentration in blood, plasma, and serum is the presence of Dnase I that may affect the amount, quality, and stability of ccf-DNA. Dnase I is a pancreatic enzyme that is present in circulation and capable of degrading free-floating DNA (17). To confirm the presence of active Dnase/s in plasma the inventors incubated 30 ng of A DNA with 40 and 80 μl of plasma for 30 min at 37° C. and measured 14% and 27% decrease of the PicoGreen signal, respectively (FIG. 5A). These data confirm the expected presence of active Dnase/s in the plasma of lung adenocarcinoma patients. Next, the inventors incubated the plasma of lung adenocarcinoma subjects with 10 mU of recombinant DNase for 30 min at 37° C. followed by detection with PicoGreen reagent. Analysis of the plasma of three lung adenocarcinoma subjects showed that an average 90% of the ccf-DNA present in plasma is DNase insensitive (FIG. 5B). Since 90% of the DNA present in plasma is protected from DNases, the inventors hypothesize that ccf-DNA is localized within lumen of EVs. It is known that serum, plasma, and liquid biopsies contain membranous EVs derived from various cell types. EVs participate in physiological and pathological processes and have potential applications in diagnostics and therapeutics. EVs are typically classified into exosomes, microvesicles and apoptotic bodies (18, 19). Since DNA in serum is DNase(s) insensitive, the inventors hypothesize that ccf-DNA in encapsulated within exosomes and/or microvesicles. Thus, the inventors isolated small EVs (exosomes and microvesicles) from 25 μl of plasma from two lung adenocarcinoma subjects (#10, 17) and one healthy control (#21). The amount of DNA was analyzed by qPCR using mt-COXIII and nu-GAPDH primers. The inventors compared the amount of DNA in untreated plasma as well as in supernatant (free-floating DNA) and pellet (isolated EVs) after EVs isolation. The inventors detected the presence of only mtDNA in both plasma and EVs since the ΔCt values obtained with nuDNA-specific primers were above 35, which supports our observation that the majority of the ccf-DNA in blood is of mitochondrial origin (FIG. 5C). Moreover, in agreement with our previous data, more than 90% of ccf-DNA in plasma is found in EVs compared to less than 10% in the supernatant (representing free-floating DNA) of analyzed subjects (FIG. 5C). These data further support our observation that free-floating DNA would be degraded by the active Dnase I present in plasma and, therefore, is protected by EVs. Most importantly, the majority of ccf-DNA within EVs is of mitochondrial origin. It can be quantified with the PicoGreen reagent as a fast and inexpensive alternative to labor and cost intensive RT-qPCR. Based on these findings, detection, profiling and quantification of ccf-mtDNA within EVs will further facilitate the identification of the source ccf-DNA due to presence of cell/tissue/organ-specific markers on EVs containing mtDNA.

High concentration of mtDNA in serum of COVID-19 samples. The investors analyzed 25 samples of COVID-19 patient's serum that included: mild (n=6), moderate (n=14) and critical (n=5). Groups were categorized based on supplemental oxygen requirement: mild, no oxygen support; moderate, face mask/nasal cannula; critical, intubated and ventilated. All analyzed serum of COVID-19 subjects were collected at the time of hospital admission while severity of SARS-CoV-2 infection was assessed later during hospitalization. As a control group, the inventors analyzed 20 samples of healthy volunteers. The amount of total DNA was quantified using PicoGreen. As showed in FIG. 6A the amount of ccf-DNA in serum of COVID-19 samples were markedly elevated. The same samples were analyzed with qPCR with mtDNA and nuDNA-specific primers. Using mtDNA (mtCOXIII) specific primers the inventors detected significant more ccf-mtDNA in serum of COVID-19 samples when compared to controls (FIG. 6B). The inventors also noticed a trend of higher amounts of nuDNA (nuACTB) in COVID-19 samples when compared to control, but it did not reach statistical significance (FIG. 6B). Importantly, the inventors measured at least 2-3 order of magnitudes difference between amount of ccf-mtDNA and nuDNA in both control and COVID-19 samples (FIGS. 6A, 6B). Based on these data the inventors concluded that: i) majority of ccf-DNA present in serum come from mitochondria (2-3 order of magnitude higher amount of mtDNA compared to nuDNA); and ii) higher amounts of mtDNA is present in the serum of COVID-19 subjects when compared to control.

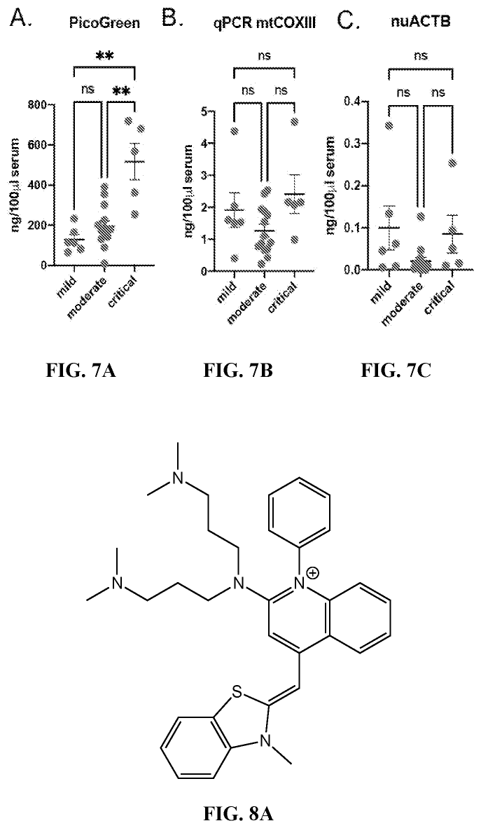

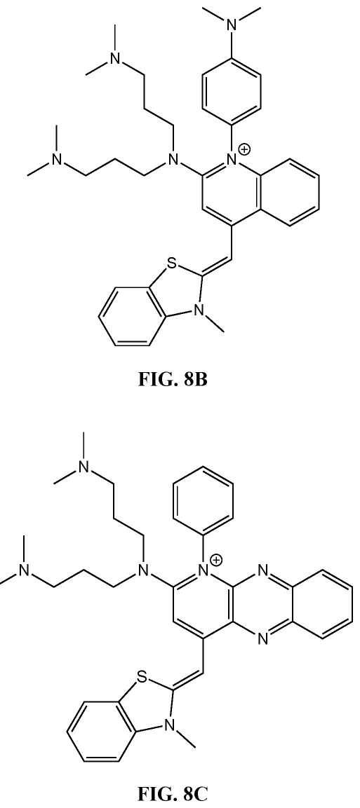

Quantification of ccf-DNA with PicoGreen reagents distinguished severity of COVID-19 samples. Using PicoGreen staining of total ccf-DNA and qPCR with mtDNA and nuDNA specific primers, the inventors analyzed serum of COVID-19 subjects. Using PicoGreen staining the inventors measured a clear trend of increasing ccf-DNA concentration in serum as a function of COVID-19 severity (FIG. 7A). In fact, despite limited number of samples, analysis showed a statistically significant difference in the amount of ccf-DNA particularly between critical and mild, moderate COVID-19 groups (FIG. 7A). In contrast, the inventors were not able to detect any statistically significant differences or trends when samples were analyzed using qPCR with mtDNA-specific primers (FIG. 7B) or nuDNA-specific primers (FIG. 7C). These data clearly demonstrate the diagnostic power of using proposed methodology with PicoGreen reagent to detect and quantify ccf-DNA in the serum of COVID-19 samples. It should be noted that samples used in these studies were obtained during hospital admission when clinical outcomes of COVID-19 progression were unknown. Thus, detection and quantification of mostly ccf-mtDNA in serum of COVID-19 patients at the time of clinical assessment can serve as a powerful early biomarker predicting disease progression, staging and monitoring therapeutic interventions. FIGS. 8A to 8C show PicoGreen (8A) and two derivatives (FIG. 8B and FIG. 8C) for use with the present invention.

Significant changes in the amount of DNA in liquid biopsies have been demonstrated in various pathologies including several types of cancer (20-24), but also various forms of trauma, such as TBI (25), stroke (26), cardiovascular diseases (27) or acute respiratory distress syndrome (28). Changes in the concentration of circulating DNA have been shown to have therapeutic and prognostic values. However, the majority of the analyses utilized RT-qPCR that is primer- and template-dependent, time consuming and not cost-effective. In this report, the inventors have developed and validated a fast and cost-effective method for quantifying the total amount of ccf-DNA in blood samples using the fluorescent PicoGreen reagent. Moreover, specific markers that are present on EVs as well as changes in EVs sizes provide additional critical information related to the source of ccf-DNA by identification of cell/tissue/organ-specific injury.

Quantification of DNA in body fluids using fluorescent probes has been proposed previously. The SYBR®Gold stain was shown to be useful for quantification of DNA contents in several bodily fluids (29). Although this study provided technical evidence of the possibility of measuring ccf-DNA in bodily fluids with fluorescent probes, it was not performed in the context of disease. More recently, the amount of ccf-DNA in subjects with breast cancer was analyzed with the SYBR®Gold fluorescent probe (30). These results show a strong correlation between the amount of ccf-DNA and the diagnosed stage of breast cancer, no direct comparisons with qPCR were drawn. The inventors use PicoGreen as fluorescent dye for the quantitative measurement of ccf-DNA in liquid biopsies. PicoGreen staining has been extensively used for histochemistry staining of both nuDNA and mtDNA. It has been used in imaging of changes of, e.g., condensed nuDNA structures with super resolution fluorescent microscopy in live time (31), or for detection of mtDNA depletion in cultured cells (32). Recently, PicoGreen staining was utilized in the quantitative investigation of DNA in plasma of mice subjected to total body irradiation. The amount of ccf-DNA correlated with the total radiation dose (33). PicoGreen detected as little as 25 pg of dsDNA/ml. In opposite to SYBR®Gold stain, PicoGreen does not detect ssDNA or RNA but only dsDNA that also provide templates for qPCR. The data herein shows a close correlation of the amount of ccf-DNA measured by PicoGreen and qPCR with mtDNA-specific primers.

One concern that has not been previously taken under consideration is the presence of active DNases in the circulation. For instance, the presence of pancreatic Dnase I has been known for long time (17). The data herein shows the presence of active DNase(s) in human plasma (FIGS. 5A to 5C). It can be assumed that active Dnase I will degrade free floating DNA and thus, the quantification of ccf-DNA may change over the time, thus the ability to rapidly, and quantitatively detect the ccf-DNA in emergency situations.

These data demonstrate that in freshly analyzed plasma samples, the major fraction of the ccf-DNA is present within the lumen of EVs and the results presented using the approach described in this invention quantify ccf-DNA that is DNase insensitive and thus does not change over the time. The inventors found that the majority of the DNA present in EVs is of mitochondrial origin (FIGS. 5A to 5C). Since enhanced oxidative stress is directly linked with most of the pathologies including cancer and trauma, the inventors hypothesize that mtDNA is specifically released from cells upon injury. The inventors have recently shown in cultured lung epithelial cells that the release of mtDNA to extracellular space via EVs occurs in also during non-cytotoxic oxidative stress conditions. (35). The diagnostic utilization of EVs in several pathologies “exploded” in recent years (36-39). However, the presence of mtDNA in EVs as a biomarker has not been carefully evaluated. In the present invention, the inventors demonstrate that DNA present in circulating EVs is of mitochondrial origin and thus it may have mitochondria-specific signatures and can be easily, rapidly, and cost-effectively quantified using PicoGreen staining. Moreover, specific markers that are present on EVs as well as changes in EVs sizes provide additional critical information related to the source of ccf-DNA by identification of cell/tissue/organ-specific injury.

Proteomics analysis of EVs isolated from blood in animals subjected to TBI. The inventors performed characterization of protein content of EVs isolated from control and TBI animals using proteomics approach. Tables 3 and 4 contain list of the protein that were found to be significantly change (amount and/or the presence) in EVs isolated from blood of animals subjected to TBI in comparison to control animals and thus can be used for staging TBI. One or more of the proteins identified in EVs that are upregulated and downregulated by TBI (Protein names (UniProt ID), in some cases the proteins are detected in the order provided, and in some cases the method detects 1, 2, 4, 5, 6, 7, 8, 9, 10, 11, 12, or 13 proteins (up or down regulation). In the case of upregulation, the method can detect 1, 2, 4, 5, 6, 7, 8, 9, 10, 11, 12, 13, 14, 15, 20, 25, 30, 35, 40, 45, 50, 60, 70, 75, 80, 90, 100, 1120, 120, or 130 proteins.

TABLE 3
Proteins found in EVs isolated from plasma to be up-regulated by TBI.

Multimerin-1 (B2RPV6); Kininogen-1; Kininogen-1 heavy chain; Bradykinin; Kininogen-
1 light chain (O08677); Proteasome subunit beta type-5 (O55234); Coagulation factor X;
Factor X light chain; Factor X heavy chain; Activated factor Xa heavy chain (O88947);
Afamin (O89020; O89020-3; O89020-2); Carbonic anhydrase 2 (P00920); Ig gamma-2A
chain C region secreted form (P01864); Hemoglobin subunit alpha (P01942); Hemoglobin
subunit beta-1; Hemoglobin subunit beta-2 (P02088; P02089); Complement factor D
(P03953-2; P03953); Complement factor B; Complement factor B Ba fragment;
Complement factor B Bb fragment (P04186); Fructose-bisphosphate aldolase A (P05064);
L-lactate dehydrogenase A chain (P06151); Complement component C9 (P06683);
Apolipoprotein A-IV (P06728); Transthyretin (P07309); Serum albumin (P07724); Alpha-
1-antitrypsin 1-1 (P07758); Serine protease inhibitor A3K (P07759); Apolipoprotein A-II;
Proapolipoprotein A-II (P09813); Major urinary protein 1 (P11588); Gelsolin (P13020-
2; P13020); Glyceraldehyde-3-phosphate dehydrogenase (P16858); Prothrombin; Activation
peptide fragment 1; Activation peptide fragment 2; Thrombin light chain; Thrombin heavy
chain (P19221); 78 kDa glucose-regulated protein; Heat shock-related 70 kDa protein 2; Heat
shock 70 kDa protein 1-like (P20029; P17156; P16627); Vitamin D-binding protein
(P21614); Alpha-1-antitrypsin 1-2 (P22599); Carboxylesterase 1C (P23953); Talin-1
(P26039); Murinoglobulin-1 (P28665); Alpha-2-HS-glycoprotein (P29699); Vitronectin
(P29788); Antithrombin-III (P32261); Leukemia inhibitory factor receptor (P42703-
2; P42703); Pyruvate kinase PKM (P52480-2; P52480); Actin, cytoplasmic 2; Actin,
cytoplasmic 2, N-terminally processed; Actin, cytoplasmic 1; Actin, cytoplasmic 1, N-
terminally processed; Actin, gamma-enteric smooth muscle; Actin, alpha skeletal muscle;
Actin, alpha cardiac muscle 1; Actin, aortic smooth muscle; Beta-actin-like protein 2
(P63260; P60710; P63268; P68134; P68033; P62737; Q8BFZ3); Apolipoprotein A-I;
Proapolipoprotein A-I; Truncated apolipoprotein A-I (Q00623); Retinol-binding protein 4
(Q00724); Alpha-1-antitrypsin 1-3 (Q00896); Alpha-1-antitrypsin 1-4 (Q00897); Alpha-1-
antitrypsin 1-5 (Q00898); Epidermal growth factor receptor (Q01279); Beta-2-glycoprotein
1 (Q01339); Serine protease inhibitor A3M (Q03734); Apolipoprotein C-II (Q05020);
Clusterin; Clusterin beta chain; Clusterin alpha chain (Q06890); Protein AMBP; Alpha-1-
microglobulin; Inter-alpha-trypsin inhibitor light chain; Trypstatin (Q07456); Complement
factor I; Complement factor I heavy chain; Complement factor I light chain (Q61129);
Ceruloplasmin (Q61147); Peroxiredoxin-2 (Q61171); Alpha-2-antiplasmin (Q61247);
Haptoglobin; Haptoglobin alpha chain; Haptoglobin beta chain (Q61646); Inter-alpha-
trypsin inhibitor heavy chain H3 (Q61704); Zinc-alpha-2-glycoprotein (Q64726);
Complement component C8 beta chain (Q8BH35; Q8BH35-2); Sulfhydryl oxidase 1
(Q8BND5-3; Q8BND5-2; Q8BND5); EGF-containing fibulin-like extracellular matrix
protein 1 (Q8BPB5); Complement C1r-A subcomponent; Complement C1r-A
subcomponent heavy chain; Complement C1r-A subcomponent light chain; Complement
C1r-B subcomponent; Complement C1r-B subcomponent heavy chain; Complement C1r-B
subcomponent light chain (Q8CG16; Q8CFG9); Complement component C8 alpha chain
(Q8K182); Complement component C8 gamma chain (Q8VCG4); Hemopexin (Q91X72);
Serotransferrin (Q921I1); Vitamin K-dependent protein Z (Q9CQW3); Carboxypeptidase N
subunit 2 (Q9DBB9); Inhibitor of carbonic anhydrase (Q9DBD0); Fetuin-B (Q9QXC1);
Hepatocyte growth factor activator; Hepatocyte growth factor activator short chain;
Hepatocyte growth factor activator long chain (Q9R098); Glycogen phosphorylase, muscle
form (Q9WUB3); Platelet factor 4 (Q9Z126); Aspartyl aminopeptidase (Q9Z2W0)

TABLE 4
Proteins found in EVs isolated from plasma to be down-regulated by TBI.

Corticosteroid-binding globulin (Q06770); Coagulation factor XIII B chain (Q07968);
Alpha-1B-glycoprotein (Q19LI2); Adiponectin (Q60994); Alpha-2-macroglobulin-P
(Q6GQT1); Coagulation factor XIII A chain (Q8BH61); Fibrinogen beta chain;
Fibrinopeptide B; Fibrinogen beta chain (Q8K0E8); Fibrinogen gamma chain (Q8VCM7);
CD5 antigen-like (Q9QWK4); Proteasome subunit alpha type-1 (Q9R1P4); Proteasome
subunit alpha type-5 (Q9Z2U1).

Methods. Human subjects. In this study the inventors used serum obtained from the Lung Cancer Biospecimen Resource Network (LCBRN). The LCBRN is a network of 3 academic medical centers, the Medical University of South Carolina (MUSC), The University of Virginia (UVA), and Washington University in St. Louis (WUSTL). Biospecimens were collected at these sites according to standard operating procedures and were shipped to the LCBRN coordination center at UVA for storage. LCBRN is an open access biorepository that provides specimens to academic and private industry scientists worldwide. The experimental protocol was approved by the UTMB Institutional Review Board (IRB), and this study was conducted in compliance with ethical and safe research practices involving human tissues. In this study the inventors also used plasma of adult patients that were admitted to level 1 trauma center from July 2011 to May 2016 at the University of Texas Health Science Center at Houston. The study was approved by the IRB. Adult patients who were admitted to our hospital and who required trauma team activation were eligible for inclusion. Patients were excluded if they were pregnant, were prisoners, were enrolled in other studies, declined to consent, or if no blood sample was drawn on admission. Consent was obtained from the patient or a legally authorized representative within 72 hours of admission or waived for patients who were discharged or died within 24 hours of hospital admission. No changes in clinical practice were implemented in this observational study. Samples were also collected at the University of Texas Medical Branch under protocol approved by the IRB. The inclusion criteria of subjects include confirmed diagnosis of primary lung cancer, chronic bronchitis or asthma, and written informed consent from subject exclusion. Exclusion criteria includes pregnant status and prisoners. Samples were also collected at the Texas Medical Branch under protocol approved for collection of biospecimens from COVID-19 subjects admitted to UTMB's hospitals. DNA isolation and qPCR Total DNA from 100 μl serum or plasma was isolated using the DNeasy Blood & Tissue Kit from Qiagen with final elution volume of 100 μl. The isolated DNA was amplified by using the Maxima SYBR Green/ROX qPCR Master Mix (Thermo Scientific) in final volume of 10 μl (4.4 μl of DNA, 0.6 μl 10 μM primers, 5 μl of master mix) with the following primers: NAD1: FW 5′-ATACCCATGGCCAACCTCCT-3′ (SEQ ID NO:1), RV 5′-GGGCCTTTGCGTAGTTGTAT-3′ (SEQ ID NO:2); COXIII: FW 5′-TGACCCACCAATCACATGC-3′ (SEQ ID NO:3), RV 5′-ATCACATGGCTAGGCCGGAG-3′ (SEQ ID NO:4); SIRT1: FW 5′-CCCGCAGCCGAGCCGCGGGG-3 (SEQ ID NO:5), RV 5-TCTTCCAACTGCCTCTCTGGCCCTCCG-3′ (SEQ ID NO:6); GAPDH: FW 5′-TGCACCACCAACTGCTTAGC-3′ (SEQ ID NO:7), RV 5′-GGCATGGACTGTGGTCATGAG-3′ (SEQ ID NO:8); ACTB: FW 5′-CATGTACGTTGCTATCCAGGC-3′ (SEQ ID NO:9), RV 5′-CTCCTTAATGTCACGCACGAT-3′ (SEQ ID NO:10). The inventors used the following thermal cycle: 95° C. for 10 minutes, 40 cycles at 95° C. for 15 seconds, and 60° C. for 1 minute. Each reaction was run in duplications. The inventors compared the expression of mtDNA-specific genes (CYTB, NAD 1, and COXIII) with the expression of the nuclear genes (SIRT1, GAPDH, and ACTB). The amount of ccf-DNA was calculated based on Ct values using isolated mtDNA and nuDNA as a DNA standard.

DNA quantification using PicoGreen reagent. Amount of total DNA in serum was measured using Quanti-iTTM PicoGreen (ThermoFisher Scientific). Briefly, 20 μl of cell free serum was mixed with 30 μl of PBS followed by addition of 50 μl of PicoGreen reagent (5 μl of Quanti-iTTM PicoGreen in 1 ml of PBS). After incubation at room temperature for 5 minutes fluorescence was measured (Ex 480/Em 520) in SpectraMax M2e spectrophotometer (Molecular Devices).

DNase treatment and isolation of EVs. For DNase treatment, 20 μl of serum was incubated with 10 mU of recombinant DNase (Qiagen) for 30 minutes at 37° C. followed by detection with PicoGreen reagent. EVs from serum were isolated using the Total Exosome Isolation Kit from serum (Invitrogen) according to manufacturer's recommendations.

Statistical analysis. All statistical analysis was performed using GraphPad Prism software. After performing a normality test, two groups were analyzed by means of a parametric or nonparametric Mann-Whitney t Test. In the analysis of more than two groups, after performing normality test, a One-Way ANOVA Kruskal-Wallis test was used. Significance differences are denoted as: *p<0.05, **p<0.01

As embodied and broadly described herein, an aspect of the present disclosure relates to a method for detecting mtDNA in a biological sample comprising, consisting essentially of, or consisting of: obtaining the biological sample; and detecting the presence of mtDNA in the sample by staining EVs in the same with a DNA staining dye. In one aspect, the DNA staining dye is PicoGreen. In another aspect, the biological sample is a liquid biopsy. In another aspect, the mtDNA is detected without mtDNA specific primers or template-dependence. In another aspect, the DNA is circulating cell-free DNA, or DNA in EVs. In another aspect, a DNA sample is obtained and tested in an emergency room, a sideline, a locker room, or a battlefield. In another aspect, the biological sample is obtained from a patient suspected of having a lung adenocarcinoma. In another aspect, an amount of mtDNA in the sample is increased when compared to a sample obtained from a subject not having a disease. In another aspect, the biological sample is obtained from a patient suspected of having a brain trauma, a concussion, or acute illness with a change in oxidative stress. In another aspect, the biological sample is obtained from a patient having or suspected of having SARS-CoV-2 or other virus infection. In another aspect, the biological sample is obtained from a person exposed to toxins or chemicals known to induce acute injury such as respiratory failure or chemical burn. In another aspect, the biological sample is obtained from a patient suffering from drug overdose. In another aspect, the biological sample is from a subject suspected of having a TBI and one or more proteins in the isolated EVs from plasma is/are up-regulated: Multimerin-1 (B2RPV6); Kininogen-1; Kininogen-1 heavy chain; Bradykinin; Kininogen-1 light chain (O08677); Proteasome subunit beta type-5 (O55234); Coagulation factor X; Factor X light chain; Factor X heavy chain; Activated factor Xa heavy chain (O88947); Afamin (O89020; O89020-3; O89020-2); Carbonic anhydrase 2 (P00920); Ig gamma-2A chain C region secreted form (P01864); Hemoglobin subunit alpha (P01942); Hemoglobin subunit beta-1; Hemoglobin subunit beta-2 (P02088; P02089); Complement factor D (P03953-2; P03953); Complement factor B; Complement factor B Ba fragment; Complement factor B Bb fragment (P04186); Fructose-bisphosphate aldolase A (P05064); L-lactate dehydrogenase A chain (P06151); Complement component C9 (P06683); Apolipoprotein A-IV (P06728); Transthyretin (P07309); Serum albumin (P07724); Alpha-1-antitrypsin 1-1 (P07758); Serine protease inhibitor A3K (P07759); Apolipoprotein A-II; Proapolipoprotein A-II (P09813); Major urinary protein 1 (P11588); Gelsolin (P13020-2; P13020); Glyceraldehyde-3-phosphate dehydrogenase (P16858); Prothrombin; Activation peptide fragment 1; Activation peptide fragment 2; Thrombin light chain; Thrombin heavy chain (P19221); 78 kDa glucose-regulated protein; Heat shock-related 70 kDa protein 2; Heat shock 70 kDa protein 1-like (P20029; P17156; P16627); Vitamin D-binding protein (P21614); Alpha-1-antitrypsin 1-2 (P22599); Carboxylesterase 1C (P23953); Talin-1 (P26039); Murinoglobulin-1 (P28665); Alpha-2-HS-glycoprotein (P29699); Vitronectin (P29788); Antithrombin-III (P32261); Leukemia inhibitory factor receptor (P42703-2; P42703); Pyruvate kinase PKM (P52480-2; P52480); Actin, cytoplasmic 2; Actin, cytoplasmic 2, N-terminally processed; Actin, cytoplasmic 1; Actin, cytoplasmic 1, N-terminally processed; Actin, gamma-enteric smooth muscle; Actin, alpha skeletal muscle; Actin, alpha cardiac muscle 1; Actin, aortic smooth muscle; Beta-actin-like protein 2 (P63260; P60710; P63268; P68134; P68033; P62737; Q8BFZ3); Apolipoprotein A-I; Proapolipoprotein A-I; Truncated apolipoprotein A-I (Q00623); Retinol-binding protein 4 (Q00724); Alpha-1-antitrypsin 1-3 (Q00896); Alpha-1-antitrypsin 1-4 (Q00897); Alpha-1-antitrypsin 1-5 (Q00898); Epidermal growth factor receptor (Q01279); Beta-2-glycoprotein 1 (Q01339); Serine protease inhibitor A3M (Q03734); Apolipoprotein C-II (Q05020); Clusterin; Clusterin beta chain; Clusterin alpha chain (Q06890); Protein AMBP; Alpha-1-microglobulin; Inter-alpha-trypsin inhibitor light chain; Trypstatin (Q07456); Complement factor I; Complement factor I heavy chain; Complement factor I light chain (Q61129); Ceruloplasmin (Q61147); Peroxiredoxin-2 (Q61171); Alpha-2-antiplasmin (Q61247); Haptoglobin; Haptoglobin alpha chain; Haptoglobin beta chain (Q61646); Inter-alpha-trypsin inhibitor heavy chain H3 (Q61704); Zinc-alpha-2-glycoprotein (Q64726); Complement component C8 beta chain (Q8BH35; Q8BH35-2); Sulfhydryl oxidase 1 (Q8BND5-3; Q8BND5-2; Q8BND5); EGF-containing fibulin-like extracellular matrix protein 1 (Q8BPB5); Complement C1r-A subcomponent; Complement C1r-A subcomponent heavy chain; Complement C1r-A subcomponent light chain; Complement C1r-B subcomponent; Complement C1r-B subcomponent heavy chain; Complement C1r-B subcomponent light chain (Q8CG16; Q8CFG9); Complement component C8 alpha chain (Q8K182); Complement component C8 gamma chain (Q8VCG4); Hemopexin (Q91X72); Serotransferrin (Q921I1); Vitamin K-dependent protein Z (Q9CQW3); Carboxypeptidase N subunit 2 (Q9DBB9); Inhibitor of carbonic anhydrase (Q9DBD0); Fetuin-B (Q9QXC1); Hepatocyte growth factor activator; Hepatocyte growth factor activator short chain; Hepatocyte growth factor activator long chain (Q9R098); Glycogen phosphorylase, muscle form (Q9WUB3); Platelet factor 4 (Q9Z126); or Aspartyl aminopeptidase (Q9Z2W0). the biological sample is from a subject suspected of having a traumatic brain injury and one or more proteins in the isolated extracellular vesicles from plasma is/are down-regulated: Corticosteroid-binding globulin (Q06770); Coagulation factor XIII B chain (Q07968); Alpha-1B-glycoprotein (Q19LI2); Adiponectin (Q60994); Alpha-2-macroglobulin-P (Q6GQT1); Coagulation factor XIII A chain (Q8BH61); Fibrinogen beta chain; Fibrinopeptide B; Fibrinogen beta chain (Q8K0E8); Fibrinogen gamma chain (Q8VCM7); CD5 antigen-like (Q9QWK4); Proteasome subunit alpha type-1 (Q9R1P4); or Proteasome subunit alpha type-5 (Q9Z2U1).

### Example 2

### Serum Amyloid A and Mitochondrial DNA in EVs: Novel Plasma Biomarkers for Diagnosis for TBI.

An established mouse models of moderate-severe TBI was used to analyze EVs' DNA and protein content. While the overall EVs count decreased during the acute phase, there was an increased number of exosomes (CD63+) accompanied by gradual increase of microvesicles derived from microglia/macrophages (CD11b+) and astrocytes (ACSA-2+) during acute and post-acute TBI phases, respectively. Noticeably, DNA content, particularly mtDNA, exhibited an immediate elevation post-injury. Acutely, neuronal (NFL) and microglial (Iba1) markers demonstrated an increased, while the astrocyte marker (GFAP) exhibited a gradual rise following TBI. Furthermore, additional novel protein biomarkers (SAA, Hp, VWF, CFD, CBG) specific to different TBI phases were identified. Biostatistical modeling and machine learning analysis determined mitochondrial DNA and SAA as the most valuable markers for TBI detection. These findings emphasize the significance of profiling of EVs' DNA and protein content, coupled with the monitoring of EVs' dynamic release, as a novel diagnostic platform for TBI through liquid biopsies.

Current TBI diagnostics (neuro exam, imaging) are subjective, necessitating alternatives. The inventors investigated blood-borne EVs for novel TBI biomarkers, analyzing DNA and protein content. Post-TBI, distinct EVs subtypes (exosomes, microvesicles from microglia and astrocytes) exhibit phase-related release patterns. EVs DNA, particularly mitochondrial DNA, emerges as an early TBI indicator. Unbiased proteomics, coupled with biostatistical modeling and graphical machine learning, identified several protein biomarkers (SAA, Hp, VWF, CFD, CBG) for diverse TBI phases. Collectively, profiling EVs' DNA and proteins are a novel diagnostic framework for TBI detection and characterization.

The current example presents a comprehensive analysis of biophysical and DNA/protein content changes in circulating EVs at different TBI phases: acute, post-acute, and chronic. EVs' protein content was examined using targeted Western analysis for known TBI biomarkers and global unbiased proteomics approach for identification of novel biomarkers. Through biostatistical and computational analysis, including graph machine learning algorithms and protein-protein interaction networks, the inventors explored a wide range of TBI biomarkers encapsulated within EVs demonstrating the suitably of using trauma-induced changes in Serum Amyloid A and mtDNA content of EVs as emerging biomarkers for diagnosis of the acute phase of TBI.

Combined Neurological and Imaging Assessment Confirmed Moderate/Severe Model of TBI. To induce TBI the inventors used a well-established, pre-clinical, close-skull, weight drop mouse model. This model is known for inducing diffuse axonal injury without the need for prior skull manipulations, and it consistently triggers robust neuroinflammatory responses with high consistency and reproducibility [15-18]. Moreover, the impact to the cranium of unrestricted animals allows for rapid acceleration of the free-moving head and torso, closely mimicking the most frequent types of human TBIs caused by traffic accidents and falls [19].

The evaluation of TBI was performed at different time points, representing the acute, post-acute, and chronic phases post-injury compared to sham controls. The severity of TBI was assessed using a modified Neurological Severity Score, NSS [20, 21], starting at 3 hours post-injury (hpi) and extended to 30 days post injury (dpi). This assessment revealed severe TBI (NSS>8) in 6, and moderate TBI (NSS 4-7) in 2 out of 8 animals at 3 hpi. Subsequent NSS measurements demonstrated a gradual decrease in scoring, with TBI animals showing a recovery trend similar to sham animals by 30 dpi. Additionally, the inventors investigated the effects of TBI using computed tomography (CT) imaging. However, axial and sagittal images revealed only skull fractures. Moreover, calculated brain volume showed only slight increase, cerebral edema, at 30 dpi. The inventors concluded that the model successfully induces moderate to severe TBI, from which animals recover similarly to previous reports [20]. However, the data also confirmed that a combination of neurological examination and imaging testing, which is the current clinical practice, provides limited information regarding the underlying neuropathology induced by TBI, particularly at the early phases post injury. This highlights the need for further research and the development of more advanced and comprehensive diagnostic methods to better understand and monitor TBI-induced neurological alterations.

Temporal Dynamics of Circulating in Plasma EVs post TBI. The latest proposed EVs classification by the International Society for Extracellular Vesicles (ISEV) categorize these molecules into small EVs (exosomes, microvesicles), large EVs, and apoptotic bodies [22]. Since, apoptotic bodies are phagocytosed, they are not considered as potential biomarker sources [23]. Following the latest guidelines from ISEV, in this study, the inventors used terms exosomes and microvesicles only to indicate their release route (derived from multivesicular bodies or pinching of the plasma membrane) [24].

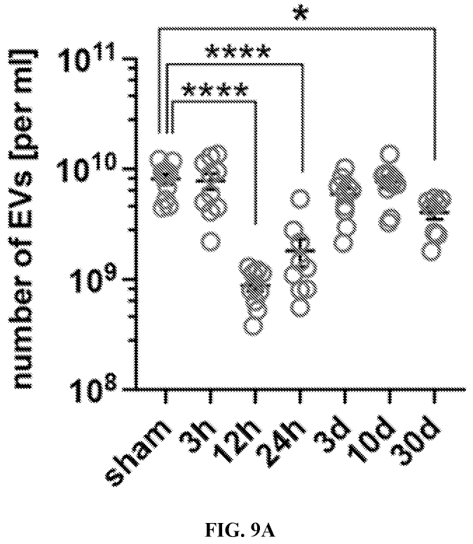

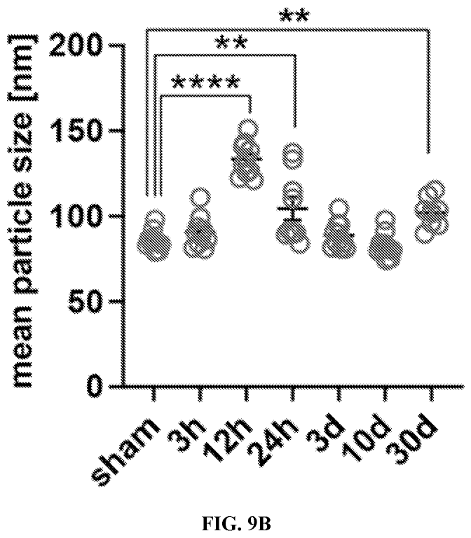

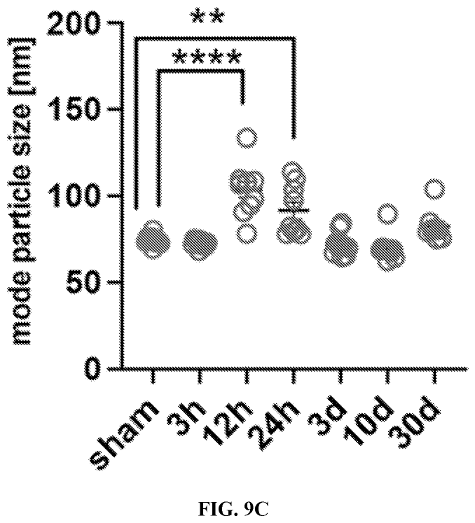

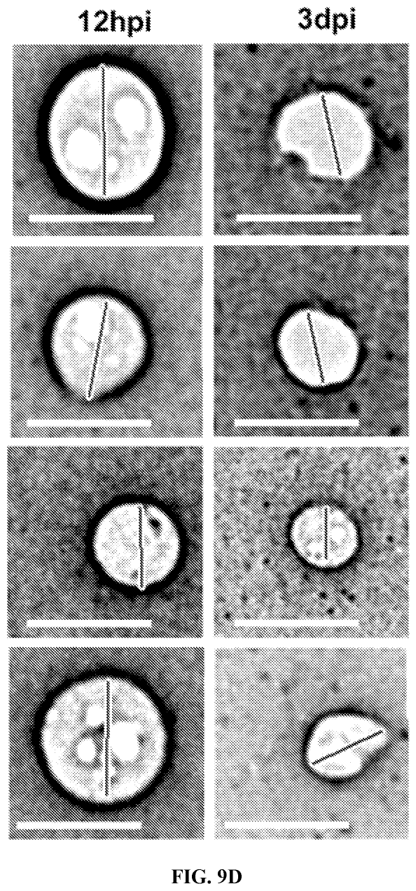

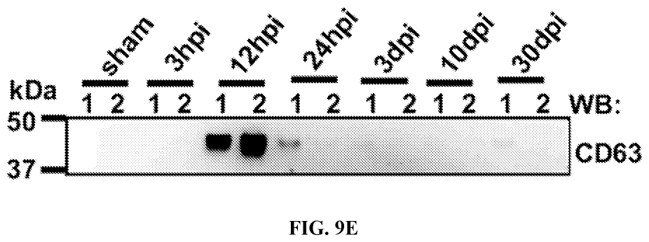

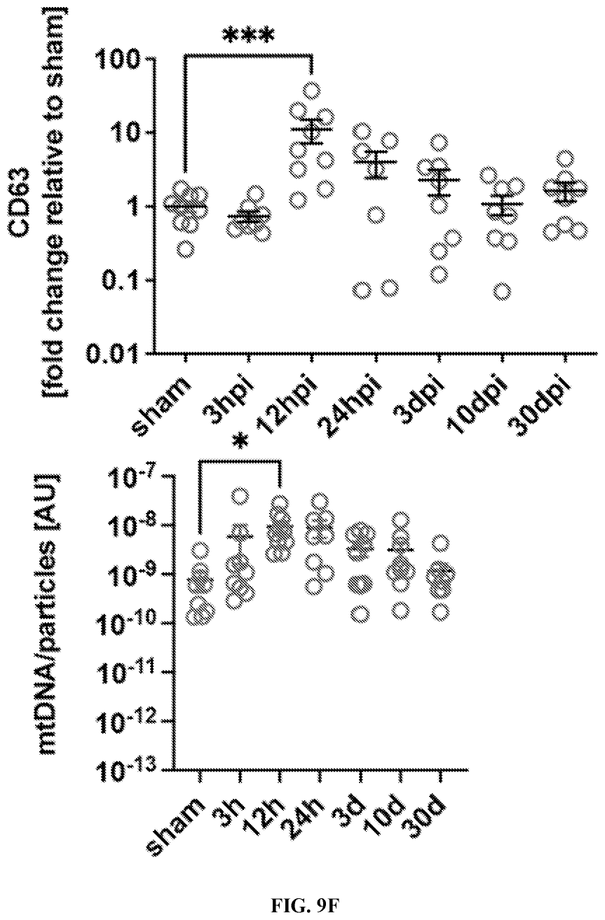

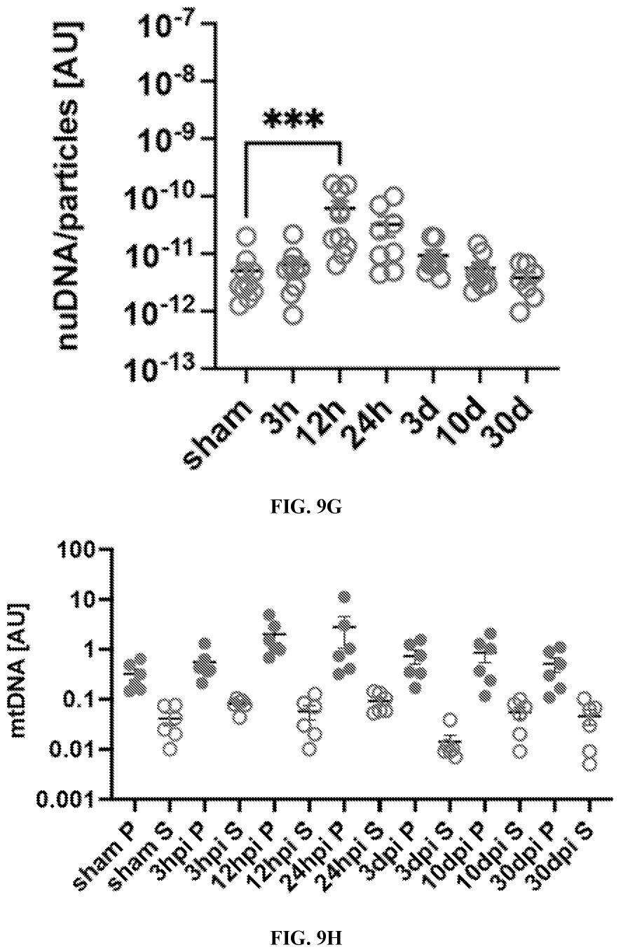

Currently, changes in the biophysical properties (number and size) of EVs in bodily fluids following TBI remain poorly characterized. To address this, the inventors isolated EVs from plasma using ultracentrifugation [25]. Nanoparticle Tracking Analysis (NTA) revealed a marked 10-fold decrease in EVs numbers at 12 hpi (8.8×10⁸/ml) and a 6-fold decrease at 24 hpi (1.8×10⁹) compared to sham controls (both ˜8×10⁹/ml, FIG. 9A and Table 5). Interestingly, EVs numbers at 3 and 10 dpi were comparable to sham controls (FIG. 9A and Table 5), but a significant decrease was observed also at 30 dpi (4.0×10⁹). Furthermore, EVs size showed significant increases at 12 and 24 hpi compared to sham controls (FIGS. 9B, C and Table 5). Transmission electron microscopy (TEM) validated the circular shape of EVs consistent with previous findings [26], and similarly as measured with NTA, EVs isolated at 12 hpi exhibited larger size compared to those at 3 dpi (FIG. 9D).

TABLE 5
Physical properties of isolated EVs following TBI.

| | sham | 3 hpi | 12 hpi | 24 hpi | 3 dpi | 10 dpi | 30 dpi |
|---|---|---|---|---|---|---|---|
| number [×10⁹/ml] | 8.1 ± 2.8 | 7.8 ± 4.0 | 0.88 ± 0.3 | 1.8 ± 1.5 | 5.8 ± 2.4 | 7.6 ± 2.9 | 4.0 ± 1.4 |
| mean size [nm] | 85.8 ± 5.6 | 90.6 ± 9.1 | 133.3 ± 9.8 | 104.4 ± 20.2 | 88.7 ± 7.8 | 82.7 ± 7.1 | 102.1 ± 8.6 |
| mode size [nm] | 74.4 ± 2.8 | 72.6 ± 2.0 | 103.6 ± 14.6 | 91.5 ± 14.6 | 72.1 ± 6.5 | 69.9 ± 7.3 | 82.5 ± 9.1 |

Considering that the majority of EVs were in the size range of 50-250 nm, consistent with small EVs (exosomes and microvesicles), our findings indicated a substantial reduction in the number but an increase in size of EVs at the acute phase post-TBI, suggesting the release of specific EV subpopulations. Thus, the inventors examined the expression of CD63, a common exosome marker [22], and observed a 10-fold increase of CD63 signal at the acute TBI phase (FIG. 9E). Notably, the decrease in EVs number and increase in EVs size were also evident at 30 dpi, suggesting changes in EVs subpopulation also during the chronic phase of TBI, reinforcing the notion that TBI represents a chronic condition rather than a singular event [27]. These data are pivotal for future investigations into EVs content at various time points post-injury, considering the changes in circulating EV numbers following TBI.

Elevated DNA Content of EVs in the Acute Phase Following TBI. In comparison to TBI-induced changes in microRNAs, the potential of measuring DNA as a TBI biomarker has received less attention. Previous studies in rat [28] and porcine [29] models of TBI demonstrated increased amounts of DNA in circulation, but specific measurements in EVs were lacking. The inventors recently reported elevated DNA levels, particularly mitochondrial DNA (mtDNA), in blood of human subjects during the acute phase post-TBI and proposed quantifying DNA as an independent indicator of TBI severity in liquid biopsies [30]. However, how DNA levels within EVs change over time post-TBI is currently unknown.

Since the inventors measured significant changes in EV numbers post-TBI (FIG. 9A), quantification of DNA content was normalized to 100 million EVs in each sample. Using qPCR with mtDNA and nuclear DNA (nuDNA)-specific primers, the inventors measured marked increase of both mtDNA and nuDNA at 12 hpi with subsequent decrease at later time points (FIGS. 9F, 9G). Furthermore, the inventors tested whether circulating plasma DNA was exclusively present in EVs or in “free floating form”. The inventors measured more than 90% of the mtDNA are found within EVs compared to “free-floating form” (FIG. 9H). Given the significantly lower nuDNA (3 order of magnitude less) than mtDNA, detecting differences in nuDNA content between EVs and the “free-floating form” was challenging due to qPCR detection limits.

These findings demonstrate that TBI triggers the release of EVs with enhanced DNA content, particularly originating from mtDNA, during the acute phase. These results support the potential of examining DNA content in EVs as an independent indicator for TBI detection. Further investigations provide valuable insights into the development of novel approaches for TBI diagnostics.

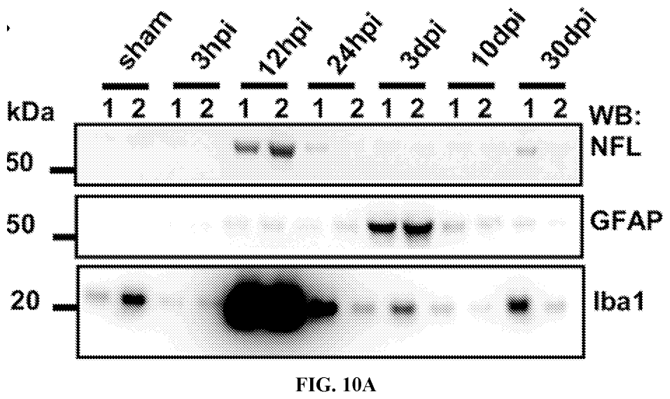

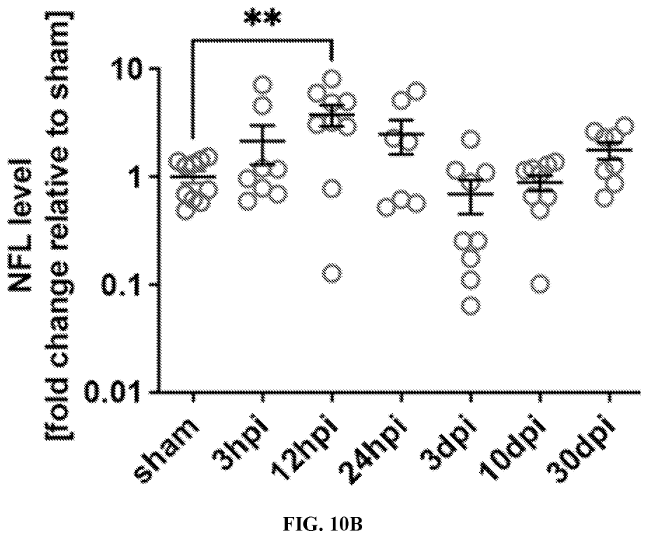

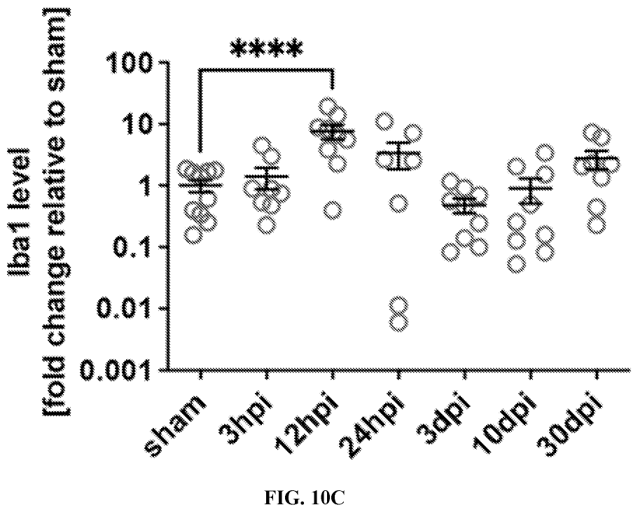

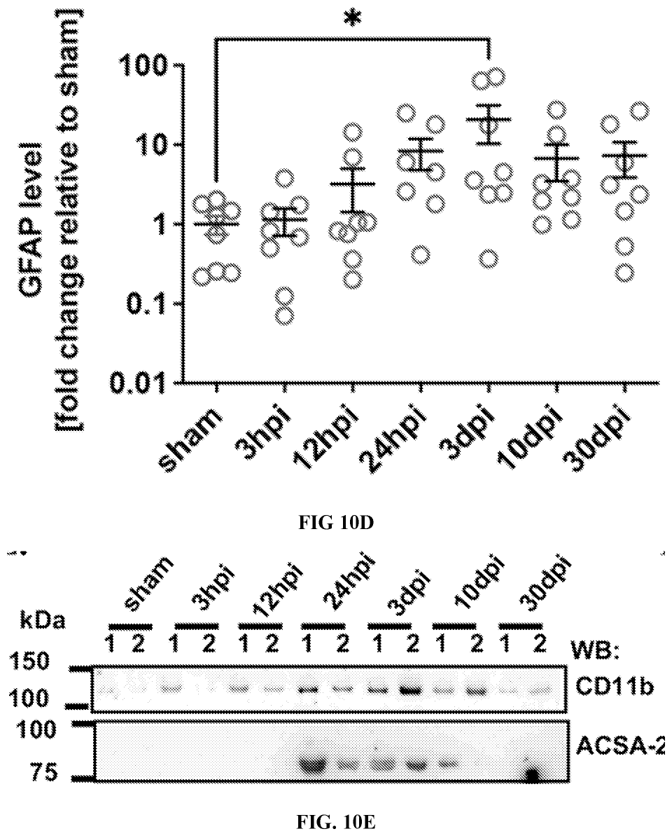

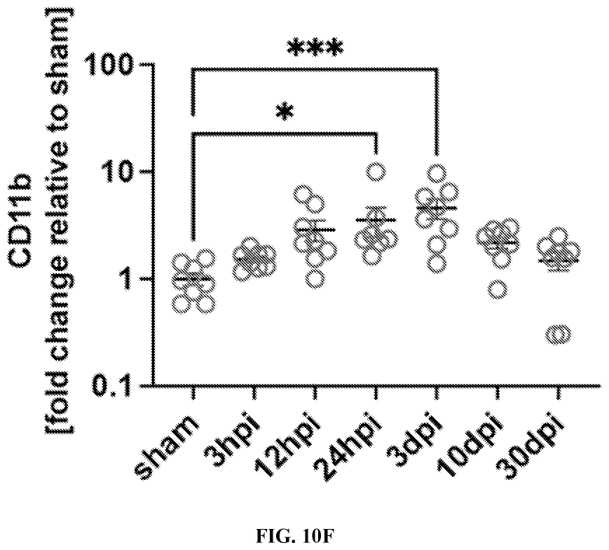

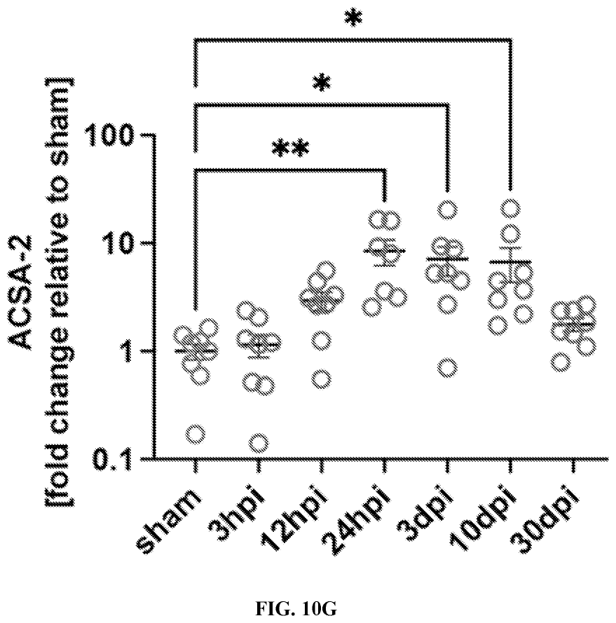

Targeted Immunoblotting for Temporal Alterations of Neuronal, Microglial, and Astrocytic Markers in Circulating EVs Post-TBI. The inventors examined established TBI-associated protein markers: GFAP, NFL, S100B, NSE, UCH-L1, and Tau [2-6], with prior EV analysis limited to NFL and GFAP [5, 7, 31-34]. Given temporal fluctuations in EV numbers post-TBI (FIG. 9A), changes in protein levels were assessed within 100 million EVs isolated at each time point post-TBI. FIG. 10A shows representative Western blots of EVs from two animals of each experimental group. These findings showed an increased presence of NFL (neuronal marker) and Iba1 (microglia marker) mainly at 12 hpi (FIGS. 10B, 10C), while GFAP (astrocyte marker) gradually increased and peaked at 3 dpi (FIG. 10D). Surprisingly, the inventors did not detect S100B, NSE, UCH-L1, and Tau proteins in the isolated EVs at any time point, although antibody specificity were confirmed using cortex homogenates. This demonstrates that these proteins are either not localized within EVs or are present in amounts below the detection limit of Western blotting. Thus, the analysis revealed cell-type-specific responses to TBI that can be measured in circulating EVs, with neuronal and microglial reactivity occurring during the acute phase while astroglial reactivity were observed in both acute and post-acute phases of TBI.

TBI led to changes in both the quantity and size of EVs circulating in plasma, signifying the immediate release of specific subpopulations of EVs in response to the injury (FIGS. 9A, 9B, 9C). Furthermore, the inventors measured an increase in levels of CD63, an exosomal marker (FIG. 9E), suggesting an enhanced release of exosomes during the acute phase of TBI. Additionally, the inventors tested the levels of CD11b (a macrophages/microglia-specific marker) and ACSA-2 (an astrocyte-specific marker) [35, 36]. Remarkably, both CD11b and ACSA-2 exhibited a gradual increase, peaking at 3 and 10 dpi, respectively (FIGS. 10E, 10F, 10G). An upregulation of glial cell reactivity, with microglia and astrocytes playing essential roles in the post-injury processes, is expected post TBI and the inventors have shown for a first time that it can be detected in liquid biopsies. Given that glial cell activation is closely associated with neuroinflammatory responses, further analysis of specific cargo within these EVs may provide valuable insights into the neuroinflammatory processes occurring in microglia and astrocytes following TBI. Thus, identifying potential neuroinflammation markers in liquid biopsies could have significant implications for diagnostic and therapeutic approaches in TBI management.

Discovery of Promising Novel TBI Markers through EVs Proteomics. Previous studies mostly focused on brain-related targets in liquid biopsies post-TBI. However, recognizing the systemic effects of TBI, the inventors conducted global unbiased proteomics analysis of 100 million EVs from each experimental timepoints. This analysis led to the identification of approximately 250 distinct proteins and among them, 229 were previously reported in the comprehensive EVs-protein-specific databases: Vesiclopedia, ExoCarta, or both. Additionally, in the current study, the inventors discovered 13 unique proteins specific to this dataset, not previously reported in EVs databases. Notably, these proteins include several major urinary proteins and subunits of complement C. These findings are not surprising, as TBI affects renal function [37], while the activation of the complement system is well reported post-TBI [38].

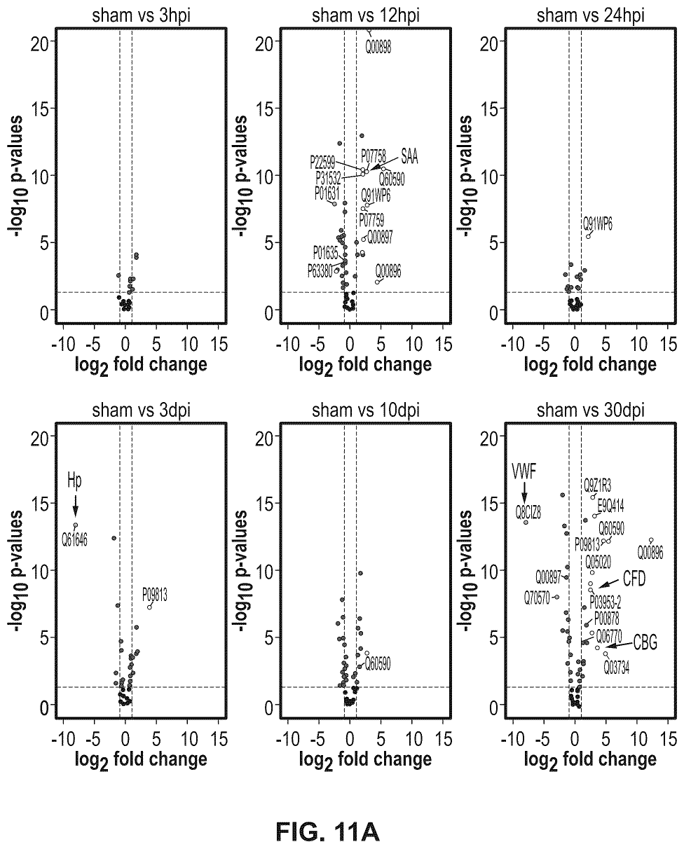

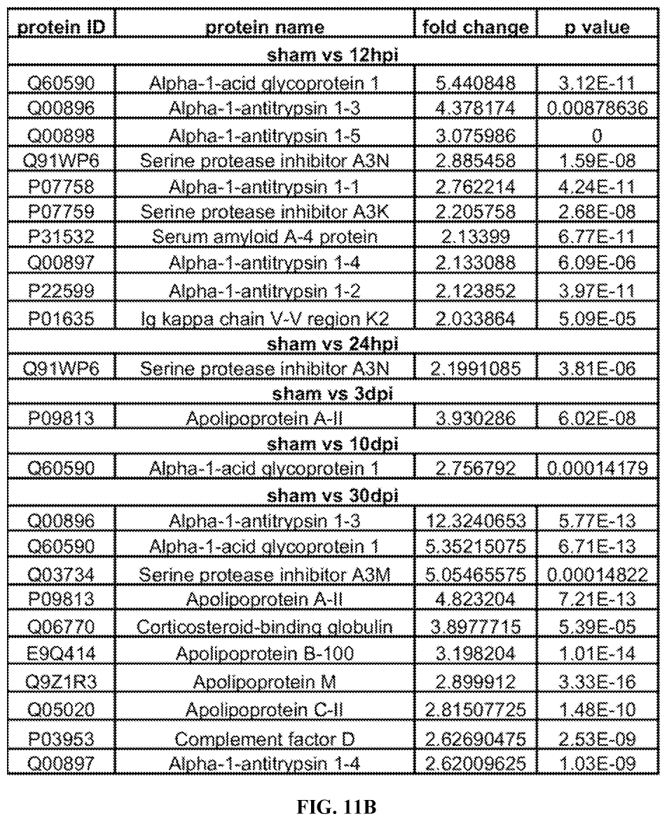

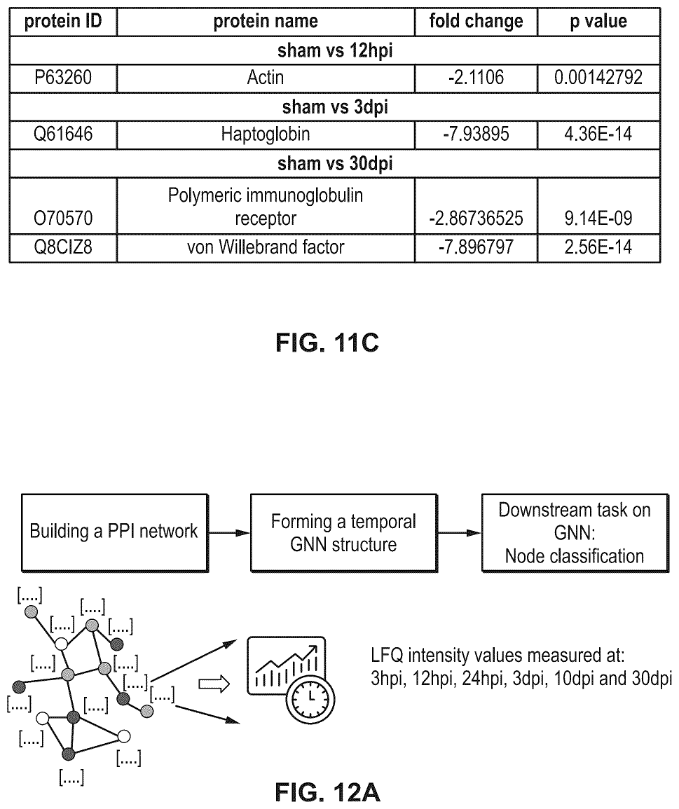

The inventors performed a linear correlation analysis using a heatmap based on the Pearson Correlation Coefficient among the LFQ (Label-Free Quantification) intensity values of each protein in the experimental data. The heatmap demonstrated high correlation within each experimental group, indicating consistency among the measurements. Next, the inventors conducted a one-way analysis of variance (ANOVA) with false discovery rate (FDR) correction [39] using each protein's LFQ intensity values. The inventors set the statistical significance level at p<0.05 and focused on proteins with fold change of LOG(FC)>2 or LOG(FC)<−2 as potential biomarkers, reflecting significant up- or downregulation, respectively. A number of identified proteins met the threshold criteria at different time points post-TBI: 8 proteins at 3 hpi, 29 at 12 hpi, 8 at 24 hpi, 14 at 3 dpi, 20 at 10 dpi, and 37 at 30 dpi (FIGS. 11A to 11C). Among these, 23 proteins were upregulated and belonged to two major groups, antitrypsin/serine protease inhibitors and apolipoproteins (FIG. 11B). These proteins were identified at multiple time points, limiting their value as specific markers for distinct phases post-TBI. However, the inventors also found specific proteins that could serve as potential novel biomarkers for acute and chronic phases post-TBI. Notably, serum amyloid A (SAA) protein was specifically upregulated for the acute phase (12 hpi), while complement factor D (CFD) and corticosteroid-binding globulin (CBG) were specific for the chronic phase (30 dpi) (FIG. 11B). Interestingly, the elevated levels of these proteins were already shown in the plasma of human TBI subjects but whether they localized with EVs was not tested [38, 40, 41]. Furthermore, the inventors identified 4 proteins that were downregulated by at least 2-fold with p<0.05 (FIG. 11B). Among these, there was a marked reduction of haptoglobin (Hp) at 3 dpi and von Willebrand factor (VWF) at 30 dpi, both known markers of vascular injury post-TBI [42].

Thus, the proteomic analysis unveiled time-dependent changes in the protein content of circulating EVs post-TBI. Among the identified proteins, SAA, CFD, CBG (upregulated), Hp, and VWF (downregulated) are particularly promising as potential novel biomarkers specific for the acute and chronic phases post-TBI.

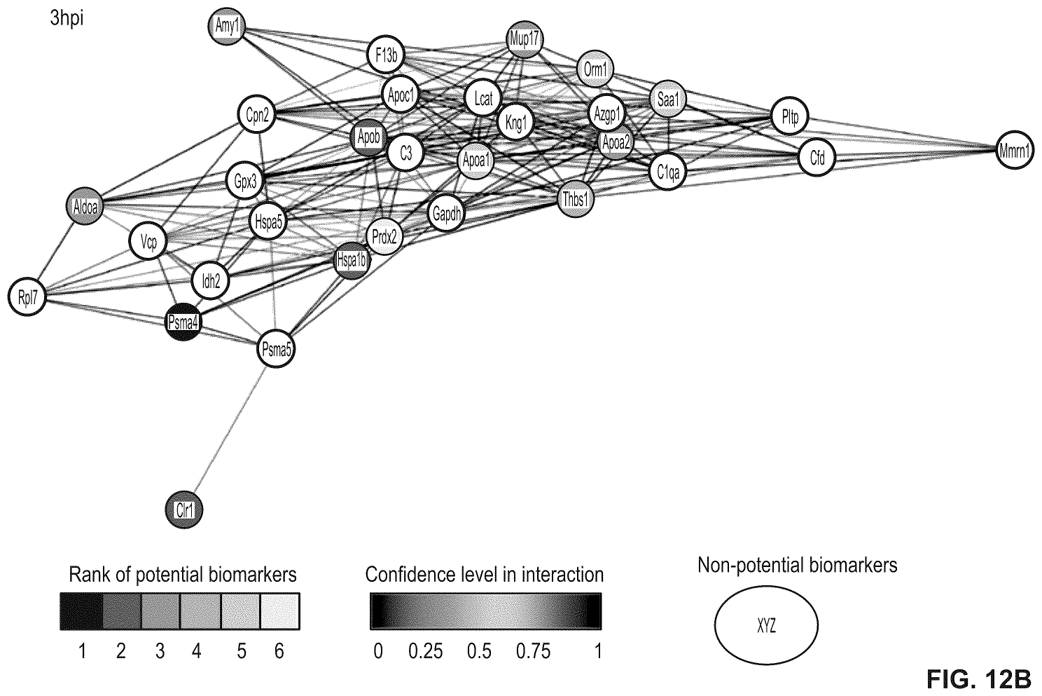

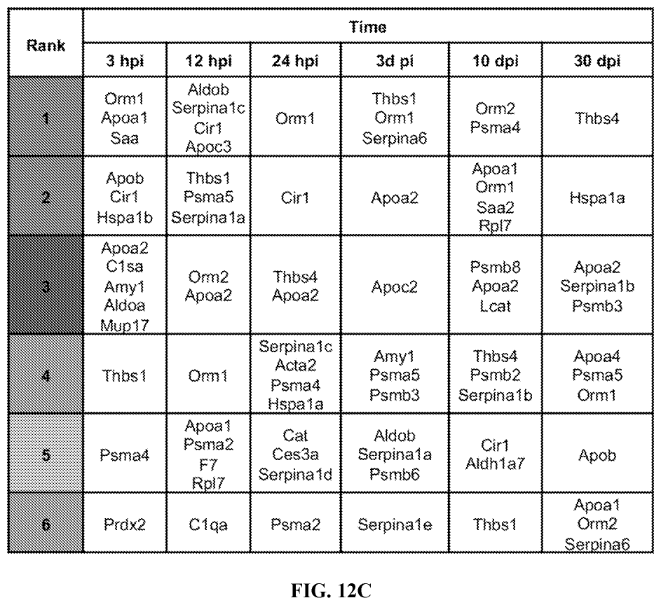

Computational Identification of TBI-specific Biomarkers. An algorithm for proteomics data was further subjected to a computational machine learning process, Graph Neural Network (GNN)-based framework, to identify potential biomarkers for TBI. The inventors constructed a Protein-Protein Interaction (PPI) network using available databases from the STRING tool [43]. The identification of potential biomarkers was formulated as a node classification problem, with node labels representing ranks from 1 to 6. Rank 1 indicated the highest probability and rank 6 the lowest probability of a protein being a potential biomarker. Analysis flow using GNN-based learning techniques predicted the labels (i.e., probabilities of individual proteins being potential biomarkers) for all nodes in the network (FIG. 12A). Generated networks with ranks for potential biomarkers for 3 hpi are shown in FIG. 12B. The inventors detected that apolipoproteins and inflammatory-response associated proteins were represented across all time points (FIG. 12C) that matched biostatistical analysis (FIGS. 11A to 11C). Most importantly, computational analysis ranked SAA as a highly probable potential biomarker for acute TBI phase and further confirmed the potential utility of SAA as a TBI biomarkers in clinical settings.

The urgent need to develop novel approaches for detecting and monitoring the detrimental effects of TBI arises from the increasing number of brain injuries. Current diagnostic methods, relying on imaging and neurological exams, are time-consuming, resource-intensive, subjective, and fail to fully capture the complex neuropathology following TBI. Ideal diagnostic markers should reflect disease status, progression, severity, potential therapeutic interventions, and readily accessible in liquid biopsies. EVs have emerged as a highly valuable source of biomarkers in various diseases, including TBI. These small vesicles are actively released by different cell types, including several brain-specific, carrying a diverse cargo of proteins, nucleic acids, and lipids that reflect the physiological and pathological state of their cellular origin. Notably, EVs can cross the BBB, facilitating the transfer of critical information from the brain to the periphery. This unique feature makes EVs accessible in peripheral blood samples, allowing for non-invasive monitoring of brain-related changes. The dynamic alterations in EV composition during TBI, such as changes in protein and DNA content, may provide valuable insights into the underlying pathophysiological processes in TBI progression. Most importantly, the inherent stability of EVs in various bodily fluids offers promise for the development of sensitive and specific biomarkers, enabling early diagnosis, prognosis, and treatment monitoring for TBI patients. In addition, changes in the biophysical properties of EVs (number, size) can provide additional valuable insights into the complex cellular responses following TBI and may open new avenues for developing EV-based biomarkers and therapeutics for TBI management.

In this study, the inventors investigated changes in biophysical properties of circulating EVs and their DNA/protein content during different phases of TBI using a weight drop mouse model. These findings revealed significant alterations in the number and size of circulating EVs during the acute TBI phase, suggesting injury-specific changes within the EV subpopulations. Specifically, the inventors observed a marked reduction in the number of EVs but an increase in their size at this stage (FIGS. 1A, 1B, 1C). Subsequently, the inventors measured an increase in the exosomal marker, CD63, during the acute phase (FIG. 1E). Furthermore, in both the acute and post-acute TBI phases, the inventors observed a dynamic increase in markers of microvesicles derived from microglia (CD11b) and astrocytes (ACSA-2) (FIGS. 2E, 2F, 2G). These data highlight the dynamic release of specific EV subpopulations post-TBI. However, there have been limited reports investigating the longitudinal changes in the biophysical properties of EVs in liquid biopsies post-TBI. In a mouse model of controlled cortical impact (CCI), an increased number of EVs was observed, accompanied by a decrease

in their size, which was measured up to 24 hpi [44]. Similarly, in human cerebrospinal fluid (CSF), an increased number of EVs was detected at 1 dpi, but changes in their size were measured after 4 dpi [45]. Discrepancies between these findings and the study might be attributed to differences in the TBI model used or the methods employed for EVs detection. Further investigation is needed to elucidate these differences. Moreover, similarly to this study, enhanced levels of microglia derived microvesicles in blood were previously detected at 24 hpi in CCI mouse model [44, 46]. No previous studies investigated the changes in the astrocyte-derived microvesicles in circulation post TBI. Nevertheless, these data underscore the critical importance of measuring the rapid dynamics of different EVs subpopulation release post-TBI.

Mitochondrial dysfunction is a key feature of TBI. However, the quantification of extracellular mtDNA in body fluids, which is a powerful indicator of mitochondrial dysfunction [47], has been relatively overlooked in TBI research. Studies have shown a significant increase in mtDNA levels in the blood during the acute phase of TBI in pigs [29], and in the cerebrospinal fluid of pediatric patients [48]. General increase in circulating cell-free (ccf) DNA has also been observed in rat plasma during the acute phase of TBI without discrimination between mtDNA and nuDNA [28]. These studies are similar to the measurements of an increased amount of particularly mtDNA in acute TBI phase (FIGS. 9F, 9G). These findings highlight the potential use of ccfDNA as an independent marker of TBI detection. The inventors observed a specific increase in mtDNA during the acute phase of TBI, with more than 90% of ccf-mtDNA found within circulating EVs (FIG. 9H). From a detection and stability perspective, this is significant because blood contains high levels of DNases I activity. The protective nature of circulating EVs enables the detection of DNA even with a limited sample volume and allows for continuous monitoring of TBI progression. This can be accomplished by classical qPCR approach or the use of a novel fluorescent-based method that the inventors have developed and validated [30].

Initially, brain-specific protein targets were explored, and biomarkers such as GFAP, NFs, S100B, NSE, UCH-L1, and Tau were proposed for independent TBI diagnosis [2-6]. However, measuring their levels in body fluids has limitations and yields conflicting results. Thus, EVs as a source of biomarkers have garnered attention in TBI research due to their intrinsic stability, ability to cross BBB and cell-type specific content reflecting cellular processes. The levels of particularly NFL and GFAP in EVs have been extensively studied [5, 7, 31-34]. Higher plasma NFL levels in exosomes isolated from TBI patients have highlighted its potential as a TBI biomarker [33, 49]. Similarly, enhanced levels of GFAP in exosomes isolated from moderate/severe TBI patients have been reported [33]. Persistent GFAP increase has also been linked to long-term cognitive deficits in TBI veterans [50, 51]. Interestingly, chronically elevated NFL levels in EVs were observed in military personnel with multiple mild TBIs [7], while elevated GFAP levels (but not NFL) were found in the civilian population with TBI [31]. The reasons for these differences, whether related to the type and frequency of TBI or the presence of GFAP/NFL in different EVs subclasses, remains unknown.

In this study, the inventors employed two approaches, targeted immunoblotting, and global proteomics, to analyze protein content of EVs in the blood. Consistent with previous findings, the inventors observed a significant increase in the levels of NFL during the acute phase of TBI (FIGS. 10A, 10B). Interestingly, NFL levels decreased during the post-acute phase (3-10 dpi) but showed an increase in the chronic phase (30 dpi) (FIGS. 10A, 10B). While the presence of the microglia marker Iba1 in EVs has not been previously reported, similar to NFL, it exhibited a marked increase during the acute TBI phase, followed by a reduction in the post-acute phase and an enhancement in the chronic TBI phase (FIGS. 10A, 10C). In contrast, the astrocyte marker GFAP displayed a different profile within EVs. The inventors observed a gradual increase in GFAP levels up to 3 dpi, and its elevated levels seemed to persist in the chronic TBI phase (FIGS. 10A, 10D). However, the inventors were unable to detect the presence of S100b, NSE, UCH-L1, and Tau in isolated EVs, despite their abundance in the mouse cortex. The inventors speculated that these proteins may exist in a “free-floating form” in the blood, making them susceptible to proteolytic activity, thereby rendering their levels unstable and leading to variability in their detection in different studies due to sample processing. Consequently, it is not surprising that most studies primarily focus on NFL and GFAP, as they are protected by the lipid layers of EVs, providing more stable and reliable measurements. Nevertheless, further investigations are warranted to better understand these findings.

It is essential to clarify that various reports have used the terms “EVs” and “exosomes” interchangeably. In accordance with the latest guidelines from the ISEV, the report adopts the nomenclature of exosomes and microvesicles to specify their origin, either derived from multivesicular bodies or plasma membrane pinching, respectively [24, 52]. The isolated EVs in this study are primarily small EVs, referred to simply as EVs, comprising a mixture of exosomes and microvesicles. However, using the widely used exosome marker CD63, the inventors observed a significant increase during the acute TBI phase, while a general decrease in the total number of EVs was observed (FIGS. 9A, 9E). Furthermore, the inventors investigated EVs derived from microglia/macrophages (CD11b) and astrocytes (ACSA-2) since microvesicles bear cell-type specific markers on their surface. The inventors found a gradual increase in these specific EV subpopulations during both the acute and post-acute TBI phases, returning to baseline levels in the chronic TBI phase (30 dpi) (FIGS. 10E, 10F, 10G). These findings support ongoing glial activation beyond the acute TBI phase. Collectively, these data strongly indicate distinct dynamics in the release of various EVs subpopulations post TBI, offering additional valuable insights. Future analyses should focus on investigating the cargo of these different EVs subpopulations, which may closely reflect cell-type specific processes following TBI.

These computational and biostatistical analysis of proteomics data identified several proteins as potential biomarkers for TBI. Apolipoproteins (Apo), alpha-acid glycoproteins (Orm), thrombospondins (Thbs) and antitrypsin (Serpina) were the most abundantly present in circulating EVs but their elevated levels could not be attributed to specific post TBI phase as they were present in all tested phases post TBI. However, the inventors identified significantly increased level of serum amyloid protein A (SAA), specifically at the acute TBI phase (FIGS. 11 and 12). SAA are small proteins with interesting association with acute phase response to chronic inflammation [53]. The increased level of SAA in serum was detected during infection [54], rheumatoid arthritis [55], and COPD [56]. SAA have been previously identified as potential biomarkers for intracranial and extracranial clinical severity in TBI and have shown predictive value for the severity of injury [40, 58]. However, none of these limited reports identified SAA within EVs. Thus, based on these data the inventors postulated measuring the level of SAA in EVs present in circulation as a novel, independent readout for TBI identification. Finally, while most of the biomarkers identified through biostatistical and computational analyses are similar, the inventors have observed some differences between them. These disparities could be attributed to several factors. The computational analysis incorporates additional topological information in the form of the Protein-Protein Interaction (PPI) network using a Graph Neural Network (GNN)-based approach, a feature not accounted for in the biostatistical approach. Furthermore, the effectiveness of both methods could potentially be enhanced with more extensive data. For instance, employing a more comprehensive protein network database to construct the PPI network could aid in better training the predictive model. Similarly, a larger sample size might improve the confidence scores of the biostatistical results.

In this study, the inventors comprehensively analyzed the DNA and protein content of circulating EVs in blood as potential TBI biomarkers. These findings highlight the presence of DNA, particularly mtDNA, and SAA within plasma EVs as a novel, stable, and reliable target for TBI detection. Additionally, the inventors validated the potential of NFL and GFAP, along with Iba1 as a biomarker of TBI. Furthermore, the investigation revealed the dynamic release of diverse subpopulations of EVs post-TBI, including those originating from brain, as potential markers of detrimental processes occurring in brain post-TBI. Collectively, this study has reveals the significance EVs as a valuable source of novel TBI biomarkers. Moreover, this study clearly indicates that diagnostic platforms should measure biophysical properties of EVs and incorporate specific DNA and protein probes to fully assess TBI severity.

Animals and procedures. Mouse model of TBI. All animal procedures were conducted in accordance with the guidelines of the Institutional Animal Care and Use Committee at the University of Texas Medical Branch and adhered to the US National Institutes of Health guidelines. C57BL6J mice (The Jackson Laboratory, #000664), aged 10-14 weeks, were housed in a controlled environment with a 12-hour light/12-hour dark cycle at a temperature of 21-23° C., and they had free access to water and a standard chow diet. To induce TBI, a non-penetrating, closed-skull weight-drop model was employed on unrestricted mice, adapted from previously published methods [15, 16, 62]. Briefly, mice underwent general health assessments and handling for 10 minutes the day prior to the injury to minimize stress related to handling. On the day of the procedure, mice were anesthetized with 3-5% isoflurane until the righting reflex was lost, and they were immediately placed in a prone position on top of a tin foil with slits, with the cranium directly underneath a plunger with a brass disc at the end. A 150 g weight was dropped from a height of 1.5 meters. The impact from the plunger caused the mice to break through the aluminum foil barrier and undergo a 180-degree flip while falling 10 cm onto a foam cushion. After injury, mice were placed on a warm pad until they regained consciousness and attained a prone position. Sham animals underwent anesthesia without injury. At various time points post-TBI, mice were euthanized, and blood samples were collected for further analysis.

Blood plasma harvest. Mice were anesthetized with 3-5% isoflurane until the righting reflex was lost and then placed in a supine position with their head in a nose cone, with a continuous flow of isoflurane administered throughout the procedure. An incision was made in the skin from the bottom of the rib cage to expose the chest cavity, ensuring that the intrathoracic pressure remained intact. The heart was located and a 1 mL syringe with a 25G×⅝ in. needle (BD, #309626) was carefully inserted at a 45-degree angle to puncture the apex of the left ventricle. Blood plasma was then slowly extracted, and the animals were humanely euthanized by cervical dislocation followed by decapitation.

Isolation of Extracellular Vesicles (EVs) from mouse plasma. To isolate the total pool of circulating extracellular vesicles (EVs) in plasma, the inventors employed a previously established method [25]. Blood plasma was collected and mixed with an equal volume of sterile dPBS in Vacutainer K2 EDTA collection tubes (BD, #367841). The tube was inverted seven times to ensure proper mixing. All subsequent centrifugation steps were performed at 4° C. The tubes were first centrifuged for 30 minutes at 2,000×g to remove circulating cells and large cellular debris. The supernatant was carefully transferred into a fresh 1.5 mL centrifuge tube and centrifuged for 45 minutes at 12,000×g to remove any remaining large cellular debris. To pellet the EVs, the supernatant was subjected to ultracentrifugation for 3 hours at 150,000×g using the FiberLite F50L-24×1.5 Fixed-Angle Rotor (ThermoFisher). After the ultracentrifugation step, the supernatant and pellet fractions were stored at −80° C. for subsequent analysis.

By following this protocol, the inventors successfully isolated the total pool of circulating small EVs present in the plasma for further investigation and analysis.

Analysis of biophysical properties of isolated EVs. The biophysical properties (number and size) of EVs were determined using Nanoparticle Tracking Analysis (NTA) with a NanoSight NS300 instrument (Malvern Analytical), employing the following settings: camera level 7 and screen gain 10. Briefly, EVs were isolated from 100 μL of precleared plasma through ultracentrifugation and then resuspended in 1 mL of sterile dPBS. A 1:100 dilution was prepared by adding 10 μL of the resuspended pellet to 990 μL of sterile dPBS. Using a 1 mL syringe (Henke Sass Wolf #4010-200V0), the diluted sample was slowly pushed through the fluid lines, and videos of the samples were recorded for 60 seconds each for subsequent image analysis. To ensure accuracy, three technical replicates were generated for each sample by advancing 100 μL of the sample between each recording.

Measurement of DNA content of isolated EVs. Each sample volume was standardized to 100 million particles, as determined by Nanoparticle Tracking Analysis (NTA). EVs were isolated using ultracentrifugation, and the total DNA was extracted from the isolated EVs using the Qiagen DNEasy Blood and Tissue Kit (#69504), following the manufacturer's protocol. To assess the amount of mitochondrial and nuclear DNA (mtDNA and nuDNA), quantitative real-time PCR (qPCR) was performed with Maxima SYBR Green/ROX qPCR Master Mix (Thermo Scientific #K0221) and specific mouse primers. The primers used were as follows:

mtDNA Forward:
(SEQ ID NO: 11)
5′-CCC AGC TAC TAC CAT CAT TCA AGT-3′.
mtDNA Reverse:
(SEQ ID NO: 12)
5′-GAT GGT TTG GGA GAT TGG TTG ATG T-3′.
nuDNA Forward:
(SEQ ID NO: 13)
5′-TTT GCT CCT GGG CCT CCA AGT T-3′
nuDNA Reverse:
(SEQ ID NO: 14)
5′-AGC CCG TGA CTG CCA CAA ATC A-3′.

The qPCR reactions were carried out using the CFX96 Touch™ Real-Time PCR Detection System (Bio-Rad) with the following thermal cycle: 95° C. for 10 minutes, followed by 40 cycles at 95° C. for 15 seconds and 60° C. for 1 minute. Each qPCR reaction was performed in technical duplicates.

To compare the amount of DNA in EVs with that in the “free-floating” form, each sample volume (100 μl) was normalized to 100 million particles, as determined by NTA. EVs were then isolated by ultracentrifugation as described above, and the EVs pellet was resuspended in 100 μl of dPBS. A total of 100 μl of the supernatant was used for total DNA extraction and qPCR, following the same protocol as described above.

Proteomics analysis of isolated EVs. The proteomics analysis of EVs isolated from mouse plasma was performed as described previously [63-66]. Briefly, the proteins in the EVs were dissolved in 20 μL of 9M urea, were reduced with 10 mM DTT for 30 minutes, followed by alkylation with 30 mM iodoacetamide for 60 minutes in the dark. The sample was diluted 10:1 with 50 mM ammonium bicarbonate and digested with 1.0 μg trypsin for 16 h at 37° C. The digestion was stopped with 10% trifluoroacetic acid. The peptides were desalted on a reversed-phase SepPak C18 cartridge (Waters, #WAT036945) and eluted with 80% acetonitrile. The eluate was dried in a SpeedVac, and the peptides were acidified with 2% acetonitrile-0.1% trifluoroacetic acid. A nanoflow ultra-high-performance chromatography Easy nLC instrument (Thermo Fisher Scientific) was coupled to a Q Exactive mass spectrometer (Thermo Scientific) with a nanoelectrospray ion source (Thermo Scientific). Peptides were loaded onto a C18 reversed-phase column (25 cm long, 75 μm inner diameter), and separated with a linear gradient of 5-35% buffer B (100% acetonitrile in 0.1% formic acid) at a flow rate of 300 nL/min over 120 min. Mass spectrometry (MS) data were acquired using a data-dependent Top15 method dynamically choosing the most abundant precursor ions from the survey scan (400-1400 m/z) using HCD fragmentation. Survey scans were acquired at a resolution of 70,000 at m/z 400. The isolation window was set to 3 Da and fragmented with normalized collision energies of 28. The maximum ion injection times for the survey scan and MS/MS scans were 20 ms and 60 ms, respectively, and the ion target values were set to 2E6 and 1e5, respectively. Selected sequenced ions were dynamically excluded for 10 s. Data were acquired using Xcalibur software.

Mass spectra were analyzed using MaxQuant software version 1.5.2.8 using the default setting [67]. Enzyme specificity was set to trypsin, defined as C-terminal to arginine and lysine excluding proline, and a maximum of two missed cleavages was allowed. Carbamidomethylcysteine was set as a fixed modification and methionine oxidation as a variable modification. The spectra were searched with the Andromeda search engine against the mouse SWISS-PROT sequence database (containing 17,000 mouse protein entries) combined with 248 common contaminants and concatenated with the reversed versions of all sequences. Protein identification required at least one unique or razor peptide per protein group. Quantification in MaxQuant was performed using the built-in XIC-based label-free quantification (LFQ) algorithm [67]. The required false positive rate for identification was set to 1% at the peptide level and 1% at the protein level, and the minimum required peptide length was set to 6 amino acids. Contaminants, reverse identification, and proteins only identified by modified peptides were excluded from further data analysis. The ‘match between runs’ feature of MaxQuant was used to transfer identifications to other LC-MS/MS runs based on their masses and retention time (maximum deviation 0.7 min), and this was also used in quantification experiments. The MaxQuant results were further analyzed using the Perseus platform [68]. The LFQ MS intensities were log2-transformed. After filtering (at least two valid LFQ values in at least one group), the remaining missing LFQ values were imputed from a normal distribution of log2 LFQ intensity of proteins in each sample by shrinking the distribution of 0.3 of standard deviation and shifting it down by 1.8 of standard deviation. The imputation was performed only once.

Analysis of isolated EVs with Western blotting. Each EVs sample was standardized to 100 million particles, as determined by NTA. The EVs were then resuspended in Laemmli sample buffer, heated at 95° C. for 2 minutes, briefly cooled, and loaded into a NuPAGE 4-12% Bis-Tris gel (Invitrogen, #NP0322). The separated proteins were transferred to 0.2 μm nitrocellulose membranes (Cytiva Amersham Protran, #10600011). The membranes were washed in TBS-T (Tris-buffered saline containing 0.5% Tween), blocked in 5% milk TBS-T for 1 hour at room temperature, and then incubated with primary antibodies at a 1:1,000 dilution in 5% milk TBS-T overnight at 4° C. The following primary antibodies were used: Mouse anti-Iba1, #ab283319; Rabbit anti-NFL, #ab9035; Mouse anti-GFAP, #ab279290; Rabbit anti-UCH-L1, #ab108986; Rabbit anti-CD11b, ab133357; Rabbit anti-S100b, ab41548; Rabbit anti-Tau, ab254256; (all from Abeam); Rabbit anti-ACSA2, #130-123-284 (Miltenyi Biotec), Rabbit anti-CD63, #PA5-92370 (Thermo Fisher); Rabbit anti-NSE, #8171 (Cell Signaling). Subsequently, the membranes were incubated with secondary antibodies (goat anti-Rabbit HRP-linked, #7074, or horse anti-Mouse HRP-linked, Cell Signaling Technologies, #7076; both from Cell Signaling) conjugated with HRP at a 1:1,000 dilution in 5% milk TBS-T for 1 hour at room temperature. Finally, the membranes were probed with ECL reagent (Cytiva Amersham, #RPN3243) or ECL Femto reagent (SuperSignal West Femto Maximum Sensitivity Substrate; Thermo Fisher, #34095). Images were captured on G-Box (Syngene), and the signal intensity was quantified using GeneTools software (Syngene). For quantification, the signal for sham samples from each blot were averaged and arbitrarily set as 1. All experimental group values were normalized to the average value of the sham samples to determine the relative abundance of the target protein.

Analysis of isolated EVs with Transmission Electron Microscopy (TEM). Isolated EVs were visualized and characterized using TEM. The EVs' pellet was resuspended in 100 μL of dPBS, and 10 μL of the sample was further diluted with 90 μL of dPBS. Ten μL of the diluted EVs was placed onto a parafilm strip and then incubated with a graphene oxide on a holey carbon copper mesh grid (Electron Microscopy Sciences, #GOHC300Cu10) for 5 minutes. Excess sample was removed using filter paper, and the grid was subsequently incubated with 1 drop of uranyl acetate (Electron Microscopy Sciences, SKU #22400). Any excess uranyl acetate was removed using filter paper. Images of the EVs were captured using a Philips CM-100 transmission electron microscope at 60 kV, equipped with an Onus SC2001 digital camera (Gatan).

Graph Neural Network (GNN) to determine potential TBI biomarkers. Each round of proteomics data was manually refined by eliminating duplicate entries of the same proteins. This total list of proteins was uploaded into the STRING database to create a Protein-Protein Interaction (PPI) network by mapping proteomics results to existing entries within the STRING database. Each protein represents a node and the interactions between individual protein molecules constitute the links/edges of PPI network. LFQ intensity values captured over different time points are pre-processed and attributed as node features. This helps to incorporate the temporal structure among LFQ intensity values of proteins within the GNN framework. All STRING runs were performed with default setting parameters and a confidence level of 0.9. Identification of potential biomarkers was formulated as a node classification problem, where the node labels represent the ranks (between 1-6) of corresponding protein being a potential biomarker. Rank 1 represents the highest probability, and Rank 6 represents the lowest probability of a protein being a potential biomarker. This flow is illustrated in FIG. 4A. A small fraction (<5%) of nodes are labeled based on prior domain knowledge and inputs from existing databases. Once the graph is constructed, GNN-based learning techniques are implemented to predict the labels (i.e., probability of individual proteins being potential biomarkers) for all the nodes in the network. GraphSAGE was used as an inductive node embedding approach that concurrently learns both the topological structure and distribution of features for a node in its local neighborhood. The operation executed at i^{th} node embedding layer is given by eq. (1).

hᵤ⁽ⁱ⁾=f⁽ⁱ⁾(hᵤ⁽ⁱ⁻¹⁾,h_{N(u)}⁽ⁱ⁻¹⁾=g[θ_{C}⁽ⁱ⁾hᵤ⁽ⁱ⁻¹⁾+θ_{A}⁽ⁱ⁾Ã(h_{N(u)}⁽ⁱ⁻¹⁾)]

Here, hᵤ⁽ⁱ⁾ represents the node embedding of node u at i^{th} layer; Ã denotes the aggregation operation; θ_{C} and θ_{A} are the parameters of the combination and aggregation operation of GNN, respectively; N(u) describes the neighborhood of node u; and g[⋅] denotes the activation function.

Biostatistical analysis. R version 4.2.2 was used to perform the proteomics data analysis. Initially, one-way ANOVA was used to analyze the results. Then, the False Discovery Rate [39] method was performed to correct for multiple comparisons testing. For paired comparisons between the Sham control group and TBI groups, Dunnett post-hoc test was used; for comparisons between only TBI groups (excluding the control group), Tukey post-hoc test was used. Statistical significance was determined with a p-value of <0.05. Additionally, heat maps were generated by calculating the Pearson correlation coefficient among experiments. The figure legends indicate the sample sizes and statistical tests used for each experiment. Fold change (FC) values were transformed and expressed in logarithmic form from LFQ intensities of each protein resulting from unbiased global proteomics. Simply, LOG(FC)=LOG(Avg. LFQ intensity protein X in TBI group/Avg. LFQ intensity protein X in Sham Control group).

As embodied and broadly described herein, an aspect of the present disclosure relates to a method of detecting DNA comprising, consisting essentially of, or consisting of: obtaining or having obtained a biological sample; and detecting the presence of mtDNA in the sample by staining EV) in the same with a DNA staining dye, without the use of mitochondrial DNA specific primers, or by monitoring EVs sizes, quantification of EVs sizes, or EVs sizes specific markers. In one aspect, the dye is selected from at least one of GeiRed, 10 EvaGreen, SYBR, PicoGreen and derivatives, TOTO, YOYO, 8080, POPO, JOJO, LOLO, SYTOX, POPRO, 80-PRO, YO-PRO, TO-PRO, JO-PRO, PO-PRO, LO-PRO, and combinations thereof. In another aspect, the dyes are engineered for recognition and quantification of mtDNA encapsulated within EVs. In another aspect, the biological sample is a liquid biopsy. In another aspect, the biological samples is exhaled breath condensate. In another aspect, the DNA is circulating cell-free DNA, or DNA encapsulated in EVs. In another aspect, a DNA sample is obtained and tested in an emergency room, a critical care setting, an ICU, a sideline, a locker room, or a battlefield. In another aspect, a DNA sample is obtained and tested in a point of care diagnosis or an at home diagnosis. In another aspect, the biological sample is obtained from a patient suspected of having a lung adenocarcinoma. In another aspect, the biological sample is obtained from a patient suspected of having a brain trauma, a concussion, or a disease of oxidative stress. In another aspect, the biological sample is obtained from a patient suspected of having SARS-CoV-2 or other virus infection. In another aspect, an amount of mitochondrial DNA in the sample is increased when compared to a sample obtained from a subject not having a disease. In another aspect, the detection of DNA is done in conjunction with other markers of tissue injury.

As embodied and broadly described herein, an aspect of the present disclosure relates to a kit for determining an amount of mtDNA is a biological sample comprising, consisting essentially of, or consisting of: a container to obtaining the biological sample; and a reagent for detecting the presence of mtDNA, in the sample by staining EVs in the same with a DNA staining dye, wherein the presence or an increase in mtDNA is indicative of a disease or condition. In one aspect, the dye is selected from at least one of GeiRed, 10 EvaGreen, SYBR, PicoGreen and derivatives, TOTO, YOYO, 8080, POPO, JOJO, LOLO, SYTOX, POPRO, 80-PRO, YO-PRO, TO-PRO, JO-PRO, PO-PRO, LO-PRO, and combinations thereof. In another aspect, the kit further comprises primers to detect the presence of cell free nuDNA, primers to detect the presence of mtDNA, or both. In another aspect, the DNA is detected within a microfluidic device. In another aspect, the microfluidic device separates components of the biological sample prior to staining the mtDNA with the dye.

As embodied and broadly described herein, an aspect of the present disclosure relates to a method for rapid detection and quantification of tissue/organ injury, comprising, consisting essentially of, or consisting of: monitoring and quantifying ccf-DNA within EVs, monitoring and quantification of EV sizes, and EV-specific markers, in a biospecimen or a liquid biopsy to assess severity of tissue/organ injury, monitoring of disease progression, or assessment of a response to one or more therapeutic interventions.

In one embodiment, the preset invention includes a method for rapid detection and quantification of tissue/organ injury, comprising: monitoring and quantifying ccf-DNA within EVs present in a biospecimen or a liquid biopsy to assess severity of tissue/organ injury, monitoring of disease progression, or assessment of a response to one or more therapeutic interventions. The invention can use an optical based detection system that can manage the detection and processing of one or more of the biomarkers of injury disclosed in this application using optical fluid chambers suitable for integration with optical and electrical sensos. For example, the optical fluid chambers suitable for integration with optical and electrical sensors can include microfluidic devices sort the samples, specimens or biospies, while enabling optical sensing and visualization of the targeted optical signal originating from the whole sample or portion of the sample stored in arrays of microfluidic chamber. The sensing and imaging device can capture and store spectral and morphological features of the samples both spatially and temporally as needed. A single, or a combination of optical sensing and imaging modalities can be used to detect and quantify the targeted biomarkers including conventional fluorescence and time resolved fluorescence measurements, absorption, and reflection measurements and optoacoustic measurements.

A person of skill in the art would readily recognize that steps of various above-described methods can be performed by programmed computers. Herein, some embodiments are also intended to cover program storage devices, e.g., digital data storage media, which are machine or computer-readable and encode machine-executable or computer-executable programs of instructions, wherein said instructions perform some or all of the steps of said above-described methods. The program storage devices may be, e.g., digital memories, magnetic storage media such as magnetic disks and magnetic tapes, hard drives, or optically readable digital data storage media. The embodiments are also intended to cover computers programmed to perform said steps of the above-described methods.

A risk score of the present invention may be calculated with an algorithm using well-known statistical analysis techniques. Non-limiting examples of statistical analysis techniques that may be used to calculate the risk score include cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, Linear Regression or classification algorithms, Nonlinear Regression or classification algorithms, analysis of variants (ANOVA), hierarchical analysis or clustering algorithms; hierarchical algorithms using decision trees; kernel based machine algorithms such as kernel partial least squares algorithms, kernel matching pursuit algorithms, kernel Fisher's discriminate analysis algorithms, or kernel principal components analysis algorithms. In preferred embodiments, the risk score may be calculated using a random forest algorithm using the concentrations of three or more sample analytes in the panel of biomarkers. In an exemplary embodiment, the risk score is calculated as described in the examples.

The functions of the various elements shown in the figures, including any functional blocks labeled as “modules”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application-specific integrated circuit (ASIC), field-programmable gate array (FPGA), read-only memory (ROM) for storing software, random access memory (RAM), and nonvolatile storage. Other hardware, conventional and/or custom, may also be included.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and

combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

### Example 1

1. Aucamp J, Bronkhorst A J, Badenhorst C P, Pretorius P J. (2016). A historical and evolutionary perspective on the biological significance of circulating DNA and extracellular vesicles. Cell Mol Life Sci. 73 (23): 4355-81. PMID: 27652382.
2. Cabel L, Proudhon C, Mariani P, Tzanis D, Beinse G, Bieche I, Pierga J Y, Bidard F C. (2017). Circulating tumor cells and circulating tumor DNA: What surgical oncologists need to know? Eur J Surg Oncol. 43 (5): 949-62. PMID: 28185687.
3. Paulsen T, Kumar P, Koseoglu M M, Dutta A. (2018). Discoveries of Extrachromosomal Circles of DNA in Normal and Tumor Cells. Trends Genet. 34 (4): 270-8. PMID: 29329720; PMCID: PMC5881399.
4. Alix-Panabieres C, Pantel K. (2016). Clinical Applications of Circulating Tumor Cells and Circulating Tumor DNA as Liquid Biopsy. Cancer Discov. 6 (5): 479-91. PMID: 26969689.
5. Koessler T, Addeo A, Nouspikel T. (2019).

Implementing circulating tumor DNA analysis in a clinical laboratory: A user manual. Adv Clin Chem. 89 131-88. PMID: 30797468.
6. Sundling K E, Lowe A C. (2019). Circulating Tumor Cells: Overview and Opportunities in Cytology. Adv Anat Pathol. 26 (1): 56-63. PMID: 30325755.
7. Thierry A R, El Messaoudi S, Gahan P B, Anker P, Stroun M. (2016). Origins, structures, and functions of circulating DNA in oncology. Cancer Metastasis Rev. 35 (3): 347-76. PMID: 27392603; PMCID: PMC5035665.
8. Vaidyanathan R, Soon R H, Zhang P, Jiang K, Lim C T. (2018). Cancer diagnosis: from tumor to liquid biopsy and beyond. Lab Chip. 19 (1): 11-34. PMID: 30480287.
9. Gyorgy B, Hung M E, Breakefield X O, Leonard J N. (2015). Therapeutic applications of extracellular vesicles: clinical promise and open questions. Annu Rev Pharmacol Toxicol. 55 439-64. PMID: 25292428; PMCID: PMC4445965.
10. Murphy D E, de Jong O G, Brouwer M, Wood M J, Lavieu G, Schiffelers R M, Vader P. (2019). Extracellular vesicle-based therapeutics: natural versus engineered targeting and trafficking. Exp Mol Med. 51 (3): 1-12. PMID: 30872574; PMCID: PMC6418170.
11. Wiklander O P B, Brennan M A, Lotvall J, Breakefield X O, El Andaloussi S. (2019). Advances in therapeutic applications of extracellular vesicles. Sci Transl Med. 11 (492). PMID: 31092696; PMCID:PMC7104415.
12. Pathak A K, Bhutani M, Kumar S, Mohan A, Guleria R. (2006). Circulating cell-free DNA in plasma/serum of lung cancer patients as a potential screening and prognostic tool. Clin Chem. 52 (10): 1833-42. PMID: 16423903.
13. Swamp V, Rajeswari M R. (2007). Circulating (cell-free) nucleic acids—a promising, non-invasive tool for early detection of several human diseases. FEBS Lett. 581 (5): 795-9. PMID: 17289032.
14. Anker P, Lyautey J, Lederrey C, Stroun M. (2001). Circulating nucleic acids in plasma or serum. Clin Chim Acta. 313 (1-2): 143-6. PMID: 11694252.
15. Singer V L, Jones L J, Yue S T, Haugland R P. (1997). Characterization of PicoGreen reagent and development of a fluorescence-based solution assay for double-stranded DNA quantitation. Anal Biochem. 249 (2): 228-38. PMID: 9212875.
16. Wang Y, Schellenberg H, Walhorn V, Toensing K, Anselmetti D. (2017). Binding mechanism of PicoGreen to DNA characterized by magnetic tweezers and fluorescence spectroscopy. Eur Biophys J. 46 (6): 561-6. PMID: 28251265.
17. Love J D, Hewitt R R. (1979). The relationship between human serum and human pancreatic DNase I. J Biol Chem. 254 (24): 12588-94. PMID:115887.
18. Margolis L, Sadovsky Y. (2019). The biology of extracellular vesicles: The known unknowns. PLoS Biol. 17 (7): e3000363. PMID: 31318874; PMCID:PMC6667152.
19. van Niel G, D'Angelo G, Raposo G. (2018). Shedding light on the cell biology of extracellular vesicles. Nat Rev Mol Cell Biol. 19 (4): 213-28. PMID:29339798.
20. Aarthy R, Mani S, Velusami S, Sundarsingh S, Rajkumar T. (2015). Role of Circulating Cell-Free DNA in Cancers. Mol Diagn Ther. 19 (6): 339-50. PMID: 26400814.
21. Bronkhorst A J, Ungerer V, Holdenrieder S. (2019). The emerging role of cell-free DNA as a molecular marker for cancer management. Biomol Detect Quantif. 17 100087. PMID: 30923679; PMCID: PMC6425120.
22. Chen Q, Zhang Z H, Wang S, Lang J H. (2019). Circulating Cell-Free DNA or Circulating Tumor DNA in the Management of Ovarian and Endometrial Cancer. Onco Targets Ther. 12 11517-30. PMID: 31920340; PMCID: PMC6938177.
23. Kustanovich A, Schwartz R, Peretz T, Grinshpun A. (2019). Life and death of circulating cell-free DNA. Cancer Biol Ther. 20 (8): 1057-67. PMID: 30990132; PMCID: PMC6606043.
24. Parsons H A, Beaver J A, Park B H. (2016). Circulating Plasma Tumor DNA. Adv Exp Med Biol. 882 259-76. PMID: 26987539.
25. Regner A, Meirelles L D S, Ikuta N, Cecchini A, Simon D. (2018). Prognostic utility of circulating nucleic acids in acute brain injuries. Expert Rev Mol Diagn. 18 (11): 925-38. PMID: 30307786.
26. Glebova K V, Veiko N N, Nikonov A A, Porokhovnik L N, Kostuyk S V. (2018). Cell-free DNA as a biomarker in stroke: Current status, problems and perspectives. Crit Rev Clin Lab Sci. 55 (1): 55-70. PMID: 29303618.
27. Biro O, Hajas O, Nagy-Balo E, Soltesz B, Csanadi Z, Nagy B. (2018). Relationship between cardiovascular diseases and circulating cell-free nucleic acids in human plasma. Biomark Med. 12 (8): 891-905. PMID: 30025473.
28. Huang L, Chang W, Huang Y, Xu X, Yang Y, Qiu H. (2020). Prognostic value of plasma mitochondrial DNA in acute respiratory distress syndrome (ARDS): a single-center observational study. J Thorac Dis. 12 (4): 1320-8. PMID: 32395269; PMCID: PMC7212167.
29. Goldshtein H, Hausmann M J, Douvdevani A. (2009). A rapid direct fluorescent assay for cell-free DNA quantification in biological fluids. Ann Clin Biochem. 46 (Pt 6): 488-94. PMID: 19729503.
30. Agassi R, Czeiger D, Shaked G, Avriel A, Sheynin J, Lavrenkov K, Ariad S, Douvdevani A. (2015). Measurement of circulating cell-free DNA levels by a simple fluorescent test in patients with breast cancer. Am J Clin Pathol. 143 (1): 18-24. PMID: 25511138.
31. Flors C. (2013). Super-resolution fluorescence imaging of directly labelled DNA: from microscopy standards to living cells. J Microsc. 251 (1): 1-4. PMID: 23700988.
32. Ashley N, Harris D, Poulton J. (2005). Detection of mitochondrial DNA depletion in living human cells using PicoGreen staining. Exp Cell Res. 303 (2): 432-46. PMID: 15652355.
33. Zhang S B, Yang S, Vidyasagar S, Zhang M, Casey-Sawicki K, Liu C, Yin L, Zhang L, Cao Y, Tian Y, Swarts S, Fenton B M, Keng P, Zhang L, Okunieff P. (2015). PicoGreen assay of circular DNA for radiation biodosimetry. Radiat Res. 183 (2): 188-95. PMID: 25574588; PMCID: PMC4452960.
34. Fernando M R, Jiang C, Krzyzanowski G D, Ryan W L. (2017). New evidence that a large proportion of human blood plasma cell-free DNA is localized in exosomes. PLoS One. 12 (8): e0183915. PMID: 28850588; PMCID: PMC5574584.
35. Szczesny B, Marcatti M, Ahmad A, Montalbano M, Brunyanszki A, Bibli S I, Papapetropoulos A, Szabo C. (2018). Mitochondrial DNA damage and subsequent activation of Z-DNA binding protein 1 links oxidative stress to inflammation in epithelial cells. Sci Rep. 8 (1): 914. PMID: 29343810.
36. Giacomini E, Makieva S, Murdica V, Vago R, Vigano P. (2020). Extracellular vesicles as a potential diagnostic tool in assisted reproduction. Curr Opin Obstet Gynecol. 32 (3): 179-84. MID:32205524.
37. Jayaseelan V P. (2019). Emerging role of exosomes as promising diagnostic tool for cancer. Cancer Gene Ther. PMID: 31477807.
38. Tang Z, Li D, Hou S, Zhu X. (2020). The cancer exosomes: Clinical implications, applications and challenges. Int J Cancer. 146 (11): 2946-59. PMID: 31671207.
39. Tavasolian F, Moghaddam AS, Rohani F, Abdollahi E, Janzamin E, Momtazi-Borojeni A A, Moallem S A, Jamialahmadi T, Sahebkar A. (2020). Exosomes: Effectual players in rheumatoid arthritis. Autoimmun Rev. 19 (6): 102511. PMID: 32171920.

### REFERENCES

### Example 2

1. Krishnamurthy, K. and D. T. Laskowitz, Cellular and Molecular Mechanisms of Secondary Neuronal Injury following Traumatic Brain Injury, in Translational Research in Traumatic Brain Injury, D. Laskowitz and G. Grant, Editors. 2016: Boca Raton (FL).
2. Czeiter, E., et al., Blood biomarkers on admission in acute traumatic brain injury: Relations to severity, CT findings and care path in the CENTER-TBI study. EBioMedicine, 2020. 56: p. 102785.
3. Gan, Z. S., et al., Blood Biomarkers for Traumatic Brain Injury: A Quantitative Assessment of Diagnostic and Prognostic Accuracy. Front Neurol, 2019. 10: p. 446.
4. Martinez, B. I. and S. E. Stabenfeldt, Current trends in biomarker discovery and analysis tools for traumatic brain injury. J Biol Eng, 2019. 13: p. 16.
5. Mondello, S., et al., Blood-Based Protein Biomarkers for the Management of Traumatic Brain Injuries in Adults Presenting to Emergency Departments with Mild Brain Injury: A Living Systematic Review and Meta-Analysis. J Neurotrauma, 2018.
6. Thelin, E. P., et al., Serial Sampling of Serum Protein Biomarkers for Monitoring Human Traumatic Brain Injury Dynamics: A Systematic Review. Front Neurol, 2017. 8: p. 300.
7. Guedes, V. A., et al., Extracellular Vesicle Proteins and MicroRNAs as Biomarkers for Traumatic Brain Injury. Front Neurol, 2020. 11: p. 663.
8. Beard, K., D. F. Meaney, and D. Issadore, Clinical Applications of Extracellular Vesicles in the Diagnosis and Treatment of Traumatic Brain Injury. J Neurotrauma, 2020. 37(19): p. 2045-2056.
9. Beard, K., et al., Extracellular vesicles as distinct biomarker reservoirs for mild traumatic brain injury diagnosis. Brain Commun, 2021. 3(3): p. fcab151.
10. Karnati, H. K., et al., Neuronal Enriched Extracellular Vesicle Proteins as Biomarkers for Traumatic Brain Injury. J Neurotrauma, 2019. 36(7): p. 975-987.
11. Ko, J., et al., Multi-Dimensional Mapping of Brain-Derived Extracellular Vesicle MicroRNA Biomarker for Traumatic Brain Injury Diagnostics. J Neurotrauma, 2020. 37(22): p. 2424-2434.
12. Mustapic, M., et al., Plasma Extracellular Vesicles Enriched for Neuronal Origin: A Potential Window into Brain Pathologic Processes. Front Neurosci, 2017. 11: p. 278.
13. Jiang, X. C. and J. Q. Gao, Exosomes as novel bio-carriers for gene and drug delivery. Int J Pharm, 2017. 521(1-2): p. 167-175.
14. Yousefpour, P. and A. Chilkoti, Co-opting biology to deliver drugs. Biotechnol Bioeng, 2014. 111(9): p. 1699-716.
15. Kalish, B. T. and M. J. Whalen, Weight Drop Models in Traumatic Brain Injury. Methods Mol Biol, 2016. 1462: p. 193-209.
16. Kane, M. J., et al., A mouse model of human repetitive mild traumatic brain injury. J Neurosci Methods, 2012. 203(1): p. 41-9.
17. Zohar, O., et al., Closed-head minimal traumatic brain injury produces long-term cognitive deficits in mice. Neuroscience, 2003. 118(4): p. 949-55.
18. Bodnar, C. N., et al., A Systematic Review of Closed Head Injury Models of Mild Traumatic Brain Injury in Mice and Rats. J Neurotrauma, 2019. 36(11): p. 1683-1706.
19. Marmarou, A., et al., A new model of diffuse brain injury in rats. Part I: Pathophysiology and biomechanics. J Neurosurg, 1994. 80(2): p. 291-300.
20. Khalin, I., et al., A mouse model of weight-drop closed head injury: emphasis on cognitive and neurological deficiency. Neural Regen Res, 2016. 11(4): p. 630-5.
21. Tsenter, J., et al., Dynamic changes in the recovery after traumatic brain injury in mice: effect of injury severity on T2-weighted MM abnormalities, and motor and cognitive functions. J Neurotrauma, 2008. 25(4): p. 324-33.
22. van Niel, G., G. D'Angelo, and G. Raposo, Shedding light on the cell biology of extracellular vesicles. Nat Rev Mol Cell Biol, 2018. 19(4): p. 213-228.
23. Khan, N. A., et al., The evolving role of extracellular vesicles (exosomes) as biomarkers in traumatic brain injury: Clinical perspectives and therapeutic implications. Front Aging Neurosci, 2022. 14: p. 933434.
24. Thery, C., et al., Minimal information for studies of extracellular vesicles 2018 (MISEV2018): a position statement of the International Society for Extracellular Vesicles and update of the MISEV2014 guidelines. J Extracell Vesicles, 2018. 7(1): p. 1535750.
25. Thery, C., et al., Isolation and characterization of exosomes from cell culture supernatants and biological fluids. Curr Protoc Cell Biol, 2006. Chapter 3: p. Unit 3 22.
26. Kopcho, S., et al., SIV Infection Regulates Compartmentalization of Circulating Blood Plasma miRNAs within Extracellular Vesicles (EVs) and Extracellular Condensates (ECs) and Decreases EV-Associated miRNA-128. Viruses, 2023. 15(3).
27. Masel, B. E. and D. S. DeWitt, Traumatic brain injury: a disease process, not an event. J Neurotrauma, 2010. 27(8): p. 1529-40.
28. Ohayon, S., et al., Cell-free DNA as a marker for prediction of brain damage in traumatic brain injury in rats. J Neurotrauma, 2012. 29(2): p. 261-7.
29. Kilbaugh, T. J., et al., Peripheral Blood Mitochondrial DNA as a Biomarker of Cerebral Mitochondrial Dysfunction following Traumatic Brain Injury in a Porcine Model. PLoS One, 2015. 10(6): p. e0130927.
30. Marcatti, M., et al., Quantification of Circulating Cell Free Mitochondrial DNA in Extracellular Vesicles with PicoGreen in Liquid Biopsies: Fast Assessment of Disease/Trauma Severity. Cells, 2021. 10(4).
31. Flynn, S., et al., Extracellular vesicle concentrations of glial fibrillary acidic protein and neurofilament light measured 1 year after traumatic brain injury. Sci Rep, 2021. 11(1): p. 3896.
32. Kawata, K., M. Mitsuhashi, and R. Aldret, A Preliminary Report on Brain-Derived Extracellular Vesicle as Novel Blood Biomarkers for Sport-Related Concussions. Front Neurol, 2018. 9: p. 239.
33. Mondello, S., et al., Circulating Brain Injury Exosomal Proteins following Moderate-To-Severe Traumatic Brain Injury: Temporal Profile, Outcome Prediction and Therapy Implications. Cells, 2020. 9(4).
34. Winston, C. N., et al., Assessing Neuronal and Astrocyte Derived Exosomes From Individuals With Mild Traumatic Brain Injury for Markers of Neurodegeneration and Cytotoxic Activity. Front Neurosci, 2019. 13: p. 1005.
35. Batiuk, M. Y., et al., Identification of region-specific astrocyte subtypes at single cell resolution. Nat Commun, 2020. 11(1): p. 1220.
36. Prinz, M., et al., Heterogeneity of CNS myeloid cells and their roles in neurodegeneration. Nat Neurosci, 2011. 14(10): p. 1227-35.
37. De Vlieger, G. and G. Meyfroidt, Kidney Dysfunction After Traumatic Brain Injury: Pathophysiology and General Management. Neurocrit Care, 2023. 38(2): p. 504-516.
38. Hammad, A., L. Westacott, and M. Zaben, The role of the complement system in traumatic brain injury: a review. J Neuroinflammation, 2018. 15(1): p. 24.
39. Korthauer, K., et al., A practical guide to methods controlling false discoveries in computational biology. Genome Biol, 2019. 20(1): p. 118.
40. Wicker, E., et al., Serum Amyloid A Protein as a Potential Biomarker for Severity and Acute Outcome in Traumatic Brain Injury. Biomed Res Int, 2019. 2019: p. 5967816.
41. Alawieh, A., et al., Identifying the Role of Complement in Triggering Neuroinflammation after Traumatic Brain Injury. J Neurosci, 2018. 38(10): p. 2519-2532.
42. Graw, J. A., et al., Endothelial dysfunction inhibits the ability of haptoglobin to prevent hemoglobin-induced hypertension. Am J Physiol Heart Circ Physiol, 2017. 312(6): p. H1120-H1127.
43. Szklarczyk, D., et al., STRING v11: protein-protein association networks with increased coverage, supporting functional discovery in genome-wide experimental datasets. Nucleic Acids Res, 2019. 47(D1): p. D607-D613.
44. Hazelton, I., et al., Exacerbation of Acute Traumatic Brain Injury by Circulating Extracellular Vesicles. J Neurotrauma, 2018. 35(4): p. 639-651.
45. Kuharic, J., et al., Severe Traumatic Brain Injury Induces Early Changes in the Physical Properties and Protein Composition of Intracranial Extracellular Vesicles. J Neurotrauma, 2019. 36(2): p. 190-200.
46. Kumar, A., et al., Microglial-derived microparticles mediate neuroinflammation after traumatic brain injury. J Neuroinflammation, 2017. 14(1): p. 47.
47. Liao, S., et al., The fate of damaged mitochondrial DNA in the cell. Biochim Biophys Acta Mol Cell Res, 2022. 1869(5): p. 119233.
48. Walko, T. D., 3rd, et al., Cerebrospinal fluid mitochondrial DNA: a novel DAMP in pediatric traumatic brain injury. Shock, 2014. 41(6): p. 499-503.
49. Guedes, V. A., et al., Extracellular vesicle neurofilament light is elevated within the first 12-months following traumatic brain injury in a U.S military population. Sci Rep, 2022. 12(1): p. 4002.
50. Peitz, C. B., et al., Blood biomarkers of traumatic brain injury and cognitive impairment in older veterans. Neurology, 2020. 95(9): p. e1126-e1133.
51. Puffer, R. C., et al., Plasma extracellular vesicles as a source of biomarkers in traumatic brain injury. J Neurosurg, 2020. 134(6): p. 1921-1928.
52. Witwer, K. W., et al., Updating MISEV: Evolving the minimal requirements for studies of extracellular vesicles. J Extracell Vesicles, 2021. 10(14): p. e12182.
53. Sack, G. H., Jr., Serum amyloid A—a review. Mol Med, 2018. 24(1): p. 46.
54. Chae, J. J., I. Aksentijevich, and D. L. Kastner, Advances in the understanding of familial Mediterranean fever and possibilities for targeted therapy. Br J Haematol, 2009. 146(5): p. 467-78.
55. Kuroda, T., et al., Significant association between renal function and area of amyloid deposition in kidney biopsy specimens in both AA amyloidosis associated with rheumatoid arthritis and AL amyloidosis. Amyloid, 2017. 24(2): p. 123-130.
56. Bozinovski, S., et al., Serum amyloid a is a biomarker of acute exacerbations of chronic obstructive pulmonary disease. Am J Respir Crit Care Med, 2008. 177(3): p. 269-78.
57. Carabias, C. S., et al., Serum Amyloid A1 as a Potential Intracranial and Extracranial Clinical Severity Biomarker in Traumatic Brain Injury. J Intensive Care Med, 2020. 35(11): p. 1180-1195.
58. Farre-Alins, V., et al., Serum Amyloid A1/Toll-Like Receptor-4 Axis, an Important Link between Inflammation and Outcome of TBI Patients. Biomedicines, 2021. 9(6).
59. Gupte, R., et al., Sex Differences in Traumatic Brain Injury: What We Know and What We Should Know. J Neurotrauma, 2019. 36(22): p. 3063-3091.
60. Biegon, A., Considering Biological Sex in Traumatic Brain Injury. Front Neurol, 2021. 12: p. 576366.
61. Frost, R. B., et al., Prevalence of traumatic brain injury in the general adult population: a meta-analysis. Neuroepidemiology, 2013. 40(3): p. 154-9.
62. Flierl, M. A., et al., Mouse closed head injury model induced by a weight-drop device. Nat Protoc, 2009. 4(9): p. 1328-37.
63. Tian, B., et al., Efficacy of Novel Highly Specific Bromodomain-Containing Protein 4 Inhibitors in Innate Inflammation-Driven Airway Remodeling. Am J Respir Cell Mol Biol, 2019. 60(1): p. 68-83.
64. Zhao, Y., et al., Pharmacoproteomics reveal novel protective activity of

bromodomain containing 4 inhibitors on vascular homeostasis in TLR3-mediated airway remodeling. J Proteomics, 2019. 205: p. 103415.
65. Zhao, Y., et al., Systematic Analysis of Cell-Type Differences in the Epithelial Secretome Reveals Insights into the Pathogenesis of Respiratory Syncytial Virus-Induced Lower Respiratory Tract Infections. J Immunol, 2017. 198(8): p. 3345-3364.
66. Zhao, Y., et al., Quantitative Proteomics of the Endothelial Secretome Identifies RC0497 as Diagnostic of Acute Rickettsial Spotted Fever Infections. Am J Pathol, 2020. 190(2): p. 306-322.
67. Cox, J. and M. Mann, MaxQuant enables high peptide identification rates, individualized p.p.b.-range mass accuracies and proteome-wide protein quantification. Nat Biotechnol, 2008. 26(12): p. 1367-72.
68. Tyanova, S., et al., The Perseus computational platform for comprehensive analysis of (prote)omics data. Nat Methods, 2016. 13(9): p. 731-40.

What is claimed is:

1. A method for detecting mitochondrial DNA in a biological sample comprising:
obtaining the biological sample; and
detecting a presence of mitochondrial DNA in the sample by staining extracellular vesicles in the biological sample with a DNA staining dye.

2. The method of claim 1, wherein the DNA staining dye is PicoGreen.

3. The method of claim 1, wherein the biological sample is a liquid biopsy.

4. The method of claim 1, wherein the mitochondrial DNA is detected without mitochondrial DNA specific primers or template-dependence; or an amount of mitochondrial DNA in the sample is increased when compared to a sample obtained from a subject not having a disease.

5. The method of claim 1, wherein DNA is circulating cell-free DNA, or DNA in extracellular vesicles.

6. The method of claim 1, wherein the biological sample is obtained and tested in an emergency room, a sideline, a locker room, or a battlefield.

7. The method of claim 1, wherein the biological sample is obtained from:
a patient suspected of having a lung adenocarcinoma;
a patient suspected of having a brain trauma, a concussion, or acute illness with a change in oxidative stress;
a patient having or suspected of having SARS-CoV-2 or other virus infection;
a person exposed to toxins or chemicals known to induce acute injury such as respiratory failure or chemical burn; or
a person suspected of having a drug overdose.

8. The method of claim 1, wherein the biological sample is from a subject suspected of having a traumatic brain injury and one or more proteins in the extracellular vesicles from plasma are up-regulated: serum amyloid A (SAA); complement factor D (CFD), Corticosteroid-binding globulin (Q06770); Multimerin-1 (B2RPV6); Kininogen-1; Kininogen-1 heavy chain; Bradykinin; Kininogen-1 light chain (O08677); Proteasome subunit beta type-5 (O55234); Coagulation factor X; Factor X light chain; Factor X heavy chain; Activated factor Xa heavy chain (O88947); Afamin (O89020; O89020-3; O89020-2); Carbonic anhydrase 2 (P00920); Ig gamma-2A chain C region secreted form (P01864); Hemoglobin subunit alpha (P01942); Hemoglobin subunit beta-1; Hemoglobin subunit beta-2 (P02088; P02089); Complement factor D (P03953-2; P03953); Complement factor B; Complement factor B Ba fragment; Complement factor B Bb fragment (P04186); Fructose-bisphosphate aldolase A (P05064); L-lactate dehydrogenase A chain (P06151); Complement component C9 (P06683); Apolipoprotein A-IV (P06728); Transthyretin (P07309); Serum albumin (P07724); Alpha-1-antitrypsin 1-1 (P07758); Serine protease inhibitor A3K (P07759); Apolipoprotein A-II; Proapolipoprotein A-II (P09813); Major urinary protein 1 (P11588); Gelsolin (P13020-2; P13020); Glyceraldehyde-3-phosphate dehydrogenase (P16858); Prothrombin; Activation peptide fragment 1; Activation peptide fragment 2; Thrombin light chain; Thrombin heavy chain (P19221); 78 kDa glucose-regulated protein; Heat shock-related 70 kDa protein 2; Heat shock 70 kDa protein 1-like (P20029; P17156; P16627); Vitamin D-binding protein (P21614); Alpha-1-antitrypsin 1-2 (P22599); Carboxylesterase 1C (P23953); Talin-1 (P26039); Murinoglobulin-1 (P28665); Alpha-2-HS-glycoprotein (P29699); Vitronectin (P29788); Antithrombin-III (P32261); Leukemia inhibitory factor receptor (P42703-2; P42703); Pyruvate kinase PKM (P52480-2; P52480); Actin, cytoplasmic 2; Actin, cytoplasmic 2, N-terminally processed; Actin, cytoplasmic 1;Actin, cytoplasmic 1, N-terminally processed; Actin, gamma-enteric smooth muscle; Actin, alpha skeletal muscle; Actin, alpha cardiac muscle 1; Actin, aortic smooth muscle; Beta-actin-like protein 2 (P63260; P60710; P63268; P68134; P68033; P62737; Q8BFZ3); Apolipoprotein A-I; Proapolipoprotein A-I; Truncated apolipoprotein A-I (Q00623); Retinol-binding protein 4 (Q00724); Alpha-1-antitrypsin 1-3 (Q00896); Alpha-1-antitrypsin 1-4 (Q00897); Alpha-1-antitrypsin 1-5 (Q00898); Epidermal growth factor receptor (Q01279); Beta-2-glycoprotein 1 (Q01339); Serine protease inhibitor A3M (Q03734); Apolipoprotein C-II (Q05020); Clusterin; Clusterin beta chain; Clusterin alpha chain (Q06890); Protein AMBP; Alpha-1-microglobulin; Inter-alpha-trypsin inhibitor light chain; Trypstatin (Q07456); Complement factor I; Complement factor I heavy chain; Complement factor I light chain (Q61129); Ceruloplasmin (Q61147); Peroxiredoxin-2 (Q61171); Alpha-2-antiplasmin (Q61247); Haptoglobin; Haptoglobin alpha chain; Haptoglobin beta chain (Q61646); Inter-alpha-trypsin inhibitor heavy chain H3 (Q61704); Zinc-alpha-2-glycoprotein (Q64726); Complement component C8 beta chain (Q8BH35; Q8BH35-2); Sulfhydryl oxidase 1 (Q8BND5-3; Q8BND5-2; Q8BND5); EGF-containing fibulin-like extracellular matrix protein 1 (Q8BPB5); Complement C1r-A subcomponent; Complement C1r-A subcomponent heavy chain; Complement C1r-A subcomponent light chain; Complement C1r-B subcomponent; Complement C1r-B subcomponent heavy chain; Complement C1r-B subcomponent light chain (Q8CG16; Q8CFG9); Complement component C8 alpha chain (Q8K182); Complement component C8 gamma chain (Q8VCG4); Hemopexin (Q91X72); Serotransferrin (Q921I1); Vitamin K-dependent protein Z (Q9CQW3); Carboxypeptidase N subunit 2 (Q9DBB9); Inhibitor of carbonic anhydrase (Q9DBD0); Fetuin-B (Q9QXC1); Hepatocyte growth factor activator; Hepatocyte growth factor activator short chain; Hepatocyte growth factor activator long chain (Q9R098); Glycogen phosphorylase, muscle form (Q9WUB3); Platelet factor 4 (Q9Z126); or Aspartyl aminopeptidase (Q9Z2W0).

9. The method of claim 1, wherein the biological sample is from a subject suspected of having a traumatic brain injury and one or more proteins in the extracellular vesicles from plasma are down-regulated: haptoglobin (IIp); von Willebrand factor (VWF); Coagulation factor XIII B chain (Q07968); Alpha-1B-glycoprotein (Q19LI2); Adiponectin (Q60994); Alpha-2-macroglobulin-P (Q6GQT1); Coagulation factor XIII A chain (Q8BH61); Fibrinogen beta chain; Fibrinopeptide B; Fibrinogen beta chain (Q8K0E8); Fibrinogen gamma chain (Q8VCM7); CD5 antigen-like (Q9QWK4); Proteasome subunit alpha type-1 (Q9R1P4); or Proteasome subunit alpha type-5 (Q9Z2U1).

10. The method of claim 1, further comprising measuring expression levels of one or more proteins in the biological sample selected from: Orm1, Alpha-1-acid glycoprotein 1; APOA1, Apolipoprotein A1; Saa, Serum Amyloid A; Aldob, Aldolase; Serpina, Alpha-1-Antitrypsin; Cir1, Corepressor Interacting With RBPJ; Apoc3, Apolipoprotein C-III; Thbs, Thrombospondin; Psma4, Proteasome 20S Subunit Alpha 4; Hspa, HSP70; Rp17, Ribosomal Protein L7; C1, Complement C; Amy, Amylase; Mup, Major Urinary Protein; Lcat, Lecithin-Cholesterol Acyltransferase; Acta, Actin Alpha 2; F7, Coagulation Factor VII; Cat, Catalase; Ces3a, Carboxylesterase 3; Aldi, Aldehyde dehydrogenase; Prdx, Peroxiredoxinin, in the biological sample;
applying an algorithm to the measured protein expression, the algorithm generating a traumatic brain injury score based on a comparison of the measured expression levels to reference levels, wherein the algorithm is selected from a machine learning algorithm, a clustering algorithm, a support vector machine, or combinations thereof; and
identifying the subject with the higher traumatic brain injury score as having a higher clinical outcome score for traumatic brain injury.

11. The method of claim 1, further comprising measuring an amount of SAA, Hp, VWF, CFD, and CBG and using an algorithm to determine a time since a traumatic brain injury.

12. A method of detecting DNA comprising:
obtaining or having obtained a biological sample; and
detecting the presence of mitochondrial DNA in the biological sample by staining extracellular vesicles in the same with a DNA staining dye, without using mitochondrial DNA specific primers, or by monitoring and quantification of extracellular vesicles sizes, extracellular vesicles surface features and content, or extracellular vesicles sizes specific markers.

13. The method of claim 12, wherein the DNA staining dye is selected from at least one of GeiRed, 10 EvaGreen, SYBR, PicoGreen and derivatives, TOTO, YOYO, 8080, POPO, JOJO, LOLO, SYTOX, POPRO, 80-PRO, YO-PRO, TO-PRO, JO-PRO, PO-PRO, LO-PRO, and combinations thereof.

14. The method of claim 12, wherein the DNA staining dyes is engineered for recognition and quantification of mitochondrial DNA encapsulated within extracellular vesicles.

15. The method of claim 12, wherein the biological sample is a liquid biopsy, or is an exhaled breath condensate.

16. The method of claim 12, wherein DNA is circulating cell-free DNA, or DNA encapsulated in extracellular vesicles.

17. The method of claim 12, wherein a DNA sample is obtained and tested in an emergency room, a critical care setting, an ICU, a sideline, a locker room, or a battlefield, in a point of care diagnostic, or an at-home test.

18. The method of claim 12, wherein the biological sample is obtained from:
a patient suspected of having a lung adenocarcinoma;
a patient suspected of having a traumatic brain injury, a brain trauma, a concussion, or a disease of oxidative stress; or
a patient suspected of having SARS-CoV-2 or other virus infection.

19. The method of claim 12, wherein an amount of mitochondrial DNA in the sample is increased when compared to a sample obtained from a subject not having a disease.

20. The method of claim 12, wherein the detection of DNA is done in conjunction with other markers of tissue injury.

21. The method of claim 12, wherein the biological sample is from a subject suspected of having a traumatic brain injury and one or more proteins in the extracellular vesicles from plasma are up-regulated: serum amyloid A (SAA); complement factor D (CFD), Corticosteroid-binding globulin (Q06770); Multimerin-1 (B2RPV6); Kininogen-1; Kininogen-1 heavy chain; Bradykinin; Kininogen-1 light chain (O08677); Proteasome subunit beta type-5 (O55234); Coagulation factor X; Factor X light chain; Factor X heavy chain; Activated factor Xa heavy chain (O88947); Afamin (O89020; O89020-3; O89020-2); Carbonic anhydrase 2 (P00920); Ig gamma-2A chain C region secreted form (P01864); Hemoglobin subunit alpha (P01942); Hemoglobin subunit beta-1; Hemoglobin subunit beta-2 (P02088; P02089); Complement factor D (P03953-2; P03953); Complement factor B; Complement factor B Ba fragment; Complement factor B Bb fragment (P04186); Fructose-bisphosphate aldolase A (P05064); L-lactate dehydrogenase A chain (P06151); Complement component C9 (P06683); Apolipoprotein A-IV (P06728); Transthyretin (P07309); Serum albumin (P07724); Alpha-1-antitrypsin 1-1 (P07758); Serine protease inhibitor A3K (P07759); Apolipoprotein A-II; Proapolipoprotein A-II (P09813); Major urinary protein 1 (P11588); Gelsolin (P13020-2;P13020); Glyceraldehyde-3-phosphate dehydrogenase (P16858); Prothrombin; Activation peptide fragment 1; Activation peptide fragment 2; Thrombin light chain; Thrombin heavy chain (P19221); 78 kDa glucose-regulated protein; Heat shock-related 70 kDa protein 2; Heat shock 70 kDa protein 1-like (P20029; P17156; P16627); Vitamin D-binding protein (P21614); Alpha-1-antitrypsin 1-2 (P22599); Carboxylesterase 1C (P23953); Talin-1 (P26039); Murinoglobulin-1 (P28665); Alpha-2-HS-glycoprotein (P29699); Vitronectin (P29788); Antithrombin-III (P32261); Leukemia inhibitory factor receptor (P42703-2; P42703); Pyruvate kinase PKM (P52480-2; P52480); Actin, cytoplasmic 2; Actin, cytoplasmic 2, N-terminally processed; Actin, cytoplasmic 1; Actin, cytoplasmic 1, N-terminally processed; Actin, gamma-enteric smooth muscle; Actin, alpha skeletal muscle; Actin, alpha cardiac muscle 1; Actin, aortic smooth muscle; Beta-actin-like protein 2 (P63260; P60710; P63268; P68134; P68033; P62737; Q8BFZ3); Apolipoprotein A-I; Proapolipoprotein A-I; Truncated apolipoprotein A-I (Q00623); Retinol-binding protein 4 (Q00724); Alpha-1-antitrypsin 1-3 (Q00896); Alpha-1-antitrypsin 1-4 (Q00897); Alpha-1-antitrypsin 1-5 (Q00898); Epidermal growth factor receptor (Q01279); Beta-2-glycoprotein 1 (Q01339); Serine protease inhibitor A3M (Q03734); Apolipoprotein C-II (Q05020); Clusterin; Clusterin beta chain; Clusterin alpha chain (Q06890); Protein AMBP; Alpha-1-microglobulin; Inter-alpha-trypsin inhibitor light chain; Trypstatin (Q07456); Complement factor I; Complement factor I heavy chain; Complement factor I light chain (Q61129); Ceruloplasmin (Q61147); Peroxiredoxin-2 (Q61171); Alpha-2-antiplasmin (Q61247); Haptoglobin; Haptoglobin alpha chain; Haptoglobin beta chain (Q61646); Inter-alpha-trypsin inhibitor heavy chain H3 (Q61704); Zinc-alpha-2-glycoprotein (Q64726); Complement component C8 beta chain (Q8BH35; Q8BH35-2); Sulfhydryl oxidase 1 (Q8BND5-3; Q8BND5-2; Q8BND5); EGF-containing fibulin-like extracellular matrix protein 1 (Q8BPB5); Complement C1r-A subcomponent; Complement C1r-A subcomponent heavy chain; Complement C1r-A subcomponent light chain; Complement C1r-B subcomponent; Complement C1r-B subcomponent heavy chain; Complement C1r-B subcomponent light chain (Q8CG16; Q8CFG9); Complement component C8 alpha chain (Q8K182); Complement component C8 gamma chain (Q8VCG4); Hemopexin (Q91X72); Serotransferrin (Q921I1); Vitamin K-dependent protein Z (Q9CQW3); Carboxypeptidase N subunit 2 (Q9DBB9); Inhibitor of carbonic anhydrase (Q9DBD0); Fetuin-B (Q9QXC1); Hepatocyte growth factor activator; Hepatocyte growth factor activator short chain; Hepatocyte growth factor activator long chain (Q9R098); Glycogen phosphorylase, muscle form (Q9WUB3); Platelet factor 4 (Q9Z126); or Aspartyl aminopeptidase (Q9Z2W0).

22. The method of claim 12, wherein the biological sample is from a subject suspected of having a traumatic brain injury and one or more proteins in the extracellular vesicles from plasma are down-regulated: haptoglobin (Hp); von Willebrand factor (VWF); Corticosteroid-binding globulin (Q06770); Coagulation factor XIII B chain (Q07968); Alpha-1B-glycoprotein (Q19LI2); Adiponectin (Q60994); Alpha-2-macroglobulin-P (Q6GQT1); Coagulation factor XIII A chain (Q8BH61); Fibrinogen beta chain; Fibrinopeptide B; Fibrinogen beta chain (Q8K0E8); Fibrinogen gamma chain (Q8VCM7); CD5 antigen-like (Q9QWK4); Proteasome subunit alpha type-1 (Q9R1P4); or Proteasome subunit alpha type-5 (Q9Z2U1).

23. The method of claim 12, further comprising measuring expression levels of one or more proteins in the biological sample selected from: Orm1, Alpha-1-acid glycoprotein 1; APOA1, Apolipoprotein A1; Saa, Serum Amyloid A; Aldob, Aldolase; Serpina, Alpha-1-Antitrypsin; Cir1, Corepressor Interacting With RBPJ; Apoc3, Apolipoprotein C-III; Thbs, Thrombospondin; Psma4, Proteasome 20S Subunit Alpha 4; Hspa, HSP70; Rp17, Ribosomal Protein L7; C1, Complement C; Amy, Amylase; Mup, Major Urinary Protein; Lcat, Lecithin-Cholesterol Acyltransferase; Acta, Actin Alpha 2; F7, Coagulation Factor VII; Cat, Catalase; Ces3a, Carboxylesterase 3; Aldi, Aldehyde dehydrogenase; Prdx, Peroxiredoxinin, in the biological sample;
applying an algorithm to the measured protein expression, the algorithm generating a traumatic brain injury score based on a comparison of the measured expression levels to reference levels, wherein the algorithm is selected from a machine learning algorithm, a clustering algorithm, a support vector machine, or combinations thereof; and
identifying the subject with the higher traumatic brain injury score as having a higher clinical outcome score for traumatic brain injury.

24. A kit for determining an amount of mitochondrial DNA is a biological sample comprising:
a container to obtaining the biological sample; and
a reagent for detecting the presence of mitochondrial DNA, in the sample by staining extracellular vesicles in the same with a DNA staining dye, monitoring extracellular vesicles sizes, quantification of extracellular vesicles sizes, or with extracellular vesicles sizes specific markers, wherein the presence or an increase in mitochondrial DNA is indicative of a disease or condition.

25. The kit of claim 24, wherein the DNA staining dye is selected from at least one of GeiRed, 10 EvaGreen, SYBR, PicoGreen and derivatives, TOTO, YOYO, 8080, POPO, JOJO, LOLO, SYTOX, POPRO, 80-PRO, YO-PRO, TO-PRO, JO-PRO, PO-PRO, LO-PRO, and combinations thereof.

26. The kit of claim 24, further comprising primers to detect cell free nuclear DNA, primers to detect the presence of mitochondrial DNA, or both.

27. The kit of claim 24, wherein the DNA is detected within a microfluidic device, and the microfluidic device separates components of the biological sample prior to staining the mitochondrial DNA with the dye.

28. A method for rapid detection and quantification of tissue/organ injury, comprising:
monitoring and quantifying circulating cell free DNA within extracellular vesicles, monitoring and quantification of extracellular vesicles sizes, and extracellular vesicles—specific markers, in a biospecimen or a liquid biopsy to assess severity of tissue/organ injury, monitoring of disease progression, or assessment of a response to one or more therapeutic interventions.

29. A method for rapid detection and quantification of tissue/organ injury, comprising:
monitoring and quantifying a circulating cell free DNA within one or more extracellular vesicles present in a biospecimen or a liquid biopsy, wherein the biospecimen or a liquid biopsy are assessed for at least one of: severity of tissue/organ injury, monitoring of disease progression, or assessment of a response to one or more therapeutic interventions,
wherein the monitoring and quantifying uses an optical-based detector that detects and processes one or more of the markers of injury in the biospecimen or a liquid biopsy, which is in fluid communication with one or more optical or electrical sensors and one or more microfluidic lines of chambers that sort the samples for optical sensing and visualization.
